(19) United States
(12) Patent Application Publication
(10) Pub. No.: US 2009/0175876 A1
(43) Pub. Date: Jul. 9, 2009

(54) METHODS OF USING PHHLA2 TO CO-STIMULATE T-CELLS

(75) Inventors: Zeren Gao, Redmond, WA (US); Brian A. Fox, Seattle, WA (US); Steven D. Levin, Seattle, WA (US); Frederick J. Ramsdell, Bainbridge Island, WA (US); Edward D. Howard, Seattle, WA (US); Janine Bilsborough, Seattle, WA (US)

Correspondence Address: ZYMOGENETICS, INC.;INTELLECTUAL PROPERTY DEPARTMENT, 1201 EASTLAKE AVENUE EAST, SEATTLE, WA, 98102-3702, US

(73) Assignee: ZymoGenetics, Inc.

(21) Appl. No.: 12/396,091

(22) Filed: Mar. 2, 2009

Related U.S. Application Data
(63) Continuation of application No. 11/433,269, filed on May 12, 2006.
(60) Provisional application No. 60/680,478, filed on May 12, 2005.

(51) Int. Cl. A61K 39/395; C07K 14/47; C07H 21/00; C12N 15/63; C12N 5/10; C12P 21/02; C07K 16/18; A61K 38/17; C12N 5/06; A61K 31/7088; A61K 35/12

(52) U.S. Cl. 424/139.1; 530/350; 536/23.5; 536/24.5; 435/320.1; 435/325; 435/69.1; 530/387.9; 530/387.3; 514/12; 435/375; 514/44; 424/93.21

(57) ABSTRACT

The invention provides pHHLA2 co-receptor polypeptides and functional fragments, antibodies to same, isolated polynucleotides encoding same, vectors containing the polynucleotides, cells containing the vectors. Methods of making and using these co-stimulatory pHHLA2 co-receptors molecules are also disclosed.

### REFERENCE TO RELATED APPLICATIONS

The present application is a continuation of U.S. patent Ser. No. 11/433,269, filed May 12, 2006, which claims the benefit of U.S. Patent Application Ser. No. 60/680,478, filed May 12, 2005, which is herein incorporated by reference in its entirety.

### BACKGROUND OF THE INVENTION

Positive and negative costimulatory signals play critical roles in the modulation of T cell activity, and the molecules that mediate these signals have proven to be effective targets for immunomodulatory agents. Positive costimulation, in addition to T cell receptor (TCR) engagement, is required for optimal activation of naive T cells, whereas negative costimulation is believed to be required for the acquisition of immunologic tolerance to self, as well as the termination of effector T cell functions. Upon interaction with B7-1 or B7-2 on the surface of antigen-presenting cells (APC), CD28, the prototypic T cell costimulatory molecule, emits signals that promote T cell proliferation and differentiation in response to TCR engagement, while the CD28 homologue cytotoxic T lymphocyte antigen-4 (CTLA-4) mediates inhibition of T cell proliferation and effector functions (Chambers et al., Ann. Rev. Immunol., 19:565-594, 2001; Egen et al., Nature Immunol., 3:611-618, 2002).

Several new molecules with homology to the B7 family have been discovered (Abbas et al., Nat. Med., 5:1345-6, 1999; Coyle et al., Nat. Immunol., 2: 203-9, 2001; Carreno et al., Annu. Rev. Immunol., 20: 29-53, 2002; Liang et al., Curr. Opin. Immunol., 14: 384-90, 2002), and their role in T cell activation is just beginning to be elucidated. These new costimulatory ligands include, for instance, B7h2, PD-L1, PD-L2, B7-H3 and B7-H4.

B7h2 (Swallow et al., Immunity, II: 423-32, 1999), also known as B7RP-1 (Yoshinaga et al., Nature, 402: 827-32, 1999), GL50 (Ling, et al., J. Immunol., 164:1653-7, 2000), B7H2 (Wang et al., Blood, 96: 2808-13, 2000), and LICOS (Brodie et al., Curr. Biol., 10: 333-6, 2000), binds to inducible costimulator (ICOS) on activated T cells, and costimulates T cell proliferation and production of cytokines such as interleukin 4 (IL-4) and IL-10.

PD-L1 (Freeman et al., J. Exp. Med., 192: 1027-34, 2000), also known as B7-H1 in humans (Dong et al., Nat. Med., 5, 1365-9, 1999), and PD-L2 (Latchman et al., Nat. Immunol., 2: 261-8, 2001), also known as B7-DC (Tseng et al., J. Exp. Med., 193, 839-46, 2001) bind to programmed death 1 (PD-1) receptor on T and B cells, although at present the function of these interactions is controversial. Some reports have demonstrated that PD-L1 and PD-L2 have inhibitory effects on T cell responses (Freeman et al., J. Exp. Med., 192: 1027-34, 2000; Latchman et al., Nat. Immunol., 2: 261-8, 2001), while others have shown that both ligands (B7-H1 and B7-DC) positively regulate T cell proliferation and specifically enhance IL-10 or interferon gamma (IFN-.gamma.) production (Dong et al., Nat. Med., 5, 1365-9, 1999; Tseng et al., J. Exp. Med., 193, 839-46, 2001).

Finally, B7-H3 and B7-H4, both newly identified B7 homologues, bind an as yet currently unknown counter-receptor(s) on activated T cells, and are reported to enhance proliferation of CD4+ T helper (Th) cells and CD8+ cytotoxic T lymphocytes (CTLs or Tcs) and selectively enhance IFN-.gamma. expression (Chapoval et al., Nat. Immunol., 2, 269-74, 2001; Sun et al., J. Immunol., 168, 6294-7, 2002).

With the exception of PD-1 ligands, which show some expression on non-lymphoid tissues, the expression of known B7 family members is largely restricted to lymphoid cells. Collectively, these studies have revealed that B7 family members are ligands on lymphoid cells that interact with cognate receptors on lymphocytes to provide positive or negative costimulatory signals that play critical roles in the regulation of cell-mediated immune responses.

In particular, many autoimmune disorders are known to involve autoreactive T cells and autoantibodies. Agents that are capable of inhibiting or eliminating autoreactive lymphocytes without compromising the immune system's ability to defend against pathogens are highly desirable. Conversely, many cancer immunotherapies, such as adoptive immunotherapy, expand tumor-specific T cell populations and direct them to attack and kill tumor cells (Dudley et al., Science 298:850-854, 2002; Pardoll, Nature Biotech., 20:1207-1208, 2002; Egen et al., Nature Immunol., 3:611-618, 2002). Agents capable of augmenting tumor attack are highly desirable. In addition, immune responses to many different antigens (e.g., microbial antigens or tumor antigens), while detectable, are frequently of insufficient magnitude to afford protection against a disease process mediated by agents (e.g., infectious microorganisms or tumor cells) expressing those antigens. It is often desirable to administer to the patient, in conjunction with the antigen, an adjuvant that serves to enhance the immune response to the antigen in the patient. It is also desirable to inhibit normal immune responses to antigen under certain circumstances. For example, the suppression of normal immune responses in a patient receiving a transplant is desirable, and agents that exhibit such immunosuppressive activity are highly desirable.

Costimulatory signals, particularly positive costimulatory signals, also play a role in the modulation of B cell activity. For example, B cell activation and the survival of germinal center B cells require T cell-derived signals in addition to stimulation by antigen. CD40 ligand present on the surface of helper T cells interacts with CD40 on the surface of B cells, and mediates many such T-cell dependent effects in B cells. Interestingly, negative costimulatory receptors analogous to CTLA-4 have not been identified on B cells. This suggests fundamental differences may exist in the way T cells and B cells are induced to respond to antigen, which has implications for mechanisms of self-tolerance as well as the inhibition of B cell effector functions, such as antibody production. Were a functional CTLA-like molecule to be found on B cells, the finding would dramatically shift our understanding of the mechanisms of B cell stimulation. Further, the identification of such receptors could provide for the development of novel therapeutic agents capable of modulating B cell activation and antibody production, and useful in the modulation of immunologic responses.

Accordingly, there is a need in the art for the identification of additional B7 family members, and molecules derived therefrom, that have either or both a T cell costimulatory activity and/or a B cell costimulatory activity. This need is based largely on their fundamental biological importance and the therapeutic potential of agents capable of affecting their activity. Such agents capable of modulating costimulatory signals would find significant use in the modulation of immune responses, and are highly desirable.

The present invention provides such polypeptides for these and other uses that should be apparent to those skilled in the art from the teachings herein.

### DESCRIPTION OF THE INVENTION

In the description that follows, a number of terms are used extensively. The following definitions are provided to facilitate understanding of the invention.

Unless otherwise specified, “a,” “an,” “the,” and “at least one” are used interchangeably and mean one or more than one.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

The term “complement of a nucleic acid molecule” refers to a nucleic acid molecule having a complementary nucleotide sequence and reverse orientation as compared to a reference nucleotide sequence. For example, the sequence 5′ ATGCACGGG 3′ is complementary to 5′ CCCGTGCAT 3′.

The term “degenerate nucleotide sequence” denotes a sequence of nucleotides that includes one or more degenerate codons as compared to a reference nucleic acid molecule that encodes a polypeptide. Degenerate codons contain different triplets of nucleotides, but encode the same amino acid residue (i.e., GAU and GAC triplets each encode Asp). The term “structural gene” refers to a nucleic acid molecule that is transcribed into messenger RNA (mRNA), which is then translated into a sequence of amino acids characteristic of a specific polypeptide.

An “isolated nucleic acid molecule” is a nucleic acid molecule that is not integrated in the genomic DNA of an organism. For example, a DNA molecule that encodes a growth factor that has been separated from the genomic DNA of a cell is an isolated DNA molecule. Another example of an isolated nucleic acid molecule is a chemically-synthesized nucleic acid molecule that is not integrated in the genome of an organism. A nucleic acid molecule that has been isolated from a particular species is smaller than the complete DNA molecule of a chromosome from that species.

A “nucleic acid molecule construct” is a nucleic acid molecule, either single- or double-stranded, that has been modified through human intervention to contain segments of nucleic acid combined and juxtaposed in an arrangement not existing in nature.

“Complementary DNA (cDNA)” is a single-stranded DNA molecule that is formed from an mRNA template by the enzyme reverse transcriptase. Typically, a primer complementary to portions of mRNA is employed for the initiation of reverse transcription. Those skilled in the art also use the term “cDNA” to refer to a double-stranded DNA molecule consisting of such a single-stranded DNA molecule and its complementary DNA strand. The term “cDNA” also refers to a clone of a cDNA molecule synthesized from an RNA template.

A “promoter” is a nucleotide sequence that directs the transcription of a structural gene. Typically, a promoter is located in the 5′ non-coding region of a gene, proximal to the transcriptional start site of a structural gene. Sequence elements within promoters that function in the initiation of transcription are often characterized by consensus nucleotide sequences. These promoter elements include RNA polymerase binding sites, TATA sequences, CAAT sequences, differentiation-specific elements (DSEs; McGehee et al., Mol. Endocrinol. 7:551 (1993)), cyclic AMP response elements (CREs), serum response elements (SREs; Treisman, Seminars in Cancer Biol. 1:47 (1990)), glucocorticoid response elements (GREs), and binding sites for other transcription factors, such as CRE/ATF (O'Reilly et al, J. Biol. Chem. 267:19938 (1992)), AP2 (Ye et al., J. Biol. Chem. 269:25728 (1994)), SP1, cAMP response element binding protein (CREB; Loeken, Gene Expr. 3:253 (1993)) and octamer factors (see, in general, Watson et al., eds., Molecular Biology of the Gene, 4th ed. (The Benjamin/Cummings Publishing Company, Inc. 1987), and Lemaigre and Rousseau, Biochem. J. 303:1 (1994)). If a promoter is an inducible promoter, then the rate of transcription increases in response to an inducing agent. In contrast, the rate of transcription is not regulated by an inducing agent if the promoter is a constitutive promoter. Repressible promoters are also known.

A “core promoter” contains essential nucleotide sequences for promoter function, including the TATA box and start of transcription. By this definition, a core promoter may or may not have detectable activity in the absence of specific sequences that may enhance the activity or confer tissue specific activity.

An “enhancer” is a type of regulatory element that can increase the efficiency of transcription, regardless of the distance or orientation of the enhancer relative to the start site of transcription.

“Heterologous DNA” refers to a DNA molecule, or a population of DNA molecules, that does not exist naturally within a given host cell. DNA molecules heterologous to a particular host cell may contain DNA derived from the host cell species (i.e., endogenous DNA) so long as that host DNA is combined with non-host DNA (i.e., exogenous DNA). For example, a DNA molecule containing a non-host DNA segment encoding a polypeptide operably linked to a host DNA segment comprising a transcription promoter is considered to be a heterologous DNA molecule. Conversely, a heterologous DNA molecule can comprise an endogenous gene operably linked with an exogenous promoter. As another illustration, a DNA molecule comprising a gene derived from a wild-type cell is considered to be heterologous DNA if that DNA molecule is introduced into a mutant cell that lacks the wild-type gene.

A “polypeptide” is a polymer of amino acid residues joined by peptide bonds, whether produced naturally or synthetically. Polypeptides of less than about 10 amino acid residues are commonly referred to as “peptides.”

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

A peptide or polypeptide encoded by a non-host DNA molecule is a “heterologous” peptide or polypeptide.

A “cloning vector” is a nucleic acid molecule, such as a plasmid, cosmid, or bacteriophage, that has the capability of replicating autonomously in a host cell. Cloning vectors typically contain one or a small number of restriction endonuclease recognition sites that allow insertion of a nucleic acid molecule in a determinable fashion without loss of an essential biological function of the vector, as well as nucleotide sequences encoding a marker gene that is suitable for use in the identification and selection of cells transformed with the cloning vector. Marker genes typically include genes that provide tetracycline resistance or ampicillin resistance.

An “expression vector” is a nucleic acid molecule encoding a gene that is expressed in a host cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter.

A “recombinant host” is a cell that contains a heterologous nucleic acid molecule, such as a cloning vector or expression vector. In the present context, an example of a recombinant host is a cell that produces pHHLA2 from an expression vector. In contrast, pHHLA2 can be produced by a cell that is a “natural source” of pHHLA2, and that lacks an expression vector.

A “fusion protein” is a hybrid protein expressed by a nucleic acid molecule comprising nucleotide sequences of at least two genes. For example, a fusion protein can comprise at least part of a pHHLA2 polypeptide fused with a polypeptide that binds an affinity matrix. Such a fusion protein provides a means to isolate large quantities of pHHLA2 using affinity chromatography.

The term “secretory signal sequence” denotes a nucleotide sequence that encodes a peptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

An “isolated polypeptide” is a polypeptide that is essentially free from contaminating cellular components, such as carbohydrate, lipid, or other proteinaceous impurities associated with the polypeptide in nature. Typically, a preparation of isolated polypeptide contains the polypeptide in a highly purified form, i.e., at least about 80% pure, at least about 90% pure, at least about 95% pure, greater than 95% pure, or greater than 99% pure. One way to show that a particular protein preparation contains an isolated polypeptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide in alternative physical forms, such as dimers or alternatively glycosylated or derivatized forms.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

As used herein, an “activating stimulus” is a molecule that delivers an activating signal to a T cell, preferably through the antigen specific T cell receptor (TCR). The activating stimulus can be sufficient to elicit a detectable response in the T cell. Alternatively, the T cell may require co-stimulation (e.g., by a pHHLA2 co-receptor polypeptide) in order to respond detectably to the activating stimulus. Examples of activating stimuli include, without limitation, antibodies that bind to the TCR or to a polypeptide of the CD3 complex that is physically associated with the TCR on the T cell surface, alloantigens, or an antigenic peptide bound to a MHC molecule.

The term “expression” refers to the biosynthesis of a gene product. For example, in the case of a structural gene, expression involves transcription of the structural gene into mRNA and the translation of mRNA into one or more polypeptides.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a polypeptide encoded by a splice variant of an mRNA transcribed from a gene. As used herein, the term “immunomodulator” includes cytokines, stem cell growth factors, lymphotoxins, co-stimulatory molecules, hematopoietic factors, and synthetic analogs of these molecules.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of less than 10⁹ M⁻¹.

An “anti-idiotype antibody” is an antibody that binds with the variable region domain of an immunoglobulin. In the present context, an anti-idiotype antibody binds with the variable region of an anti-pHHLA2 antibody, and thus, an anti-idiotype antibody mimics an epitope of pHHLA2.

An “antibody fragment” is a portion of an antibody such as F(ab′)₂, F(ab)₂, Fab′, Fab, scFv, and the like. Regardless of structure, an antibody fragment binds with the same antigen that is recognized by the intact antibody. For example, an anti-pHHLA2 monoclonal antibody fragment binds with an epitope of the extracellular domain of pHHLA2.

The term “antibody fragment” also includes a synthetic or a genetically engineered polypeptide that binds to a specific antigen, such as polypeptides consisting of the light chain variable region, “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.

A “chimeric antibody” is a recombinant protein that contains the variable domains and complementary determining regions derived from a rodent antibody, while the remainder of the antibody molecule is derived from a human antibody.

“Humanized antibodies” are recombinant proteins in which murine complementarity determining regions of a monoclonal antibody have been transferred from heavy and light variable chains of the murine immunoglobulin into a human variable domain.

As used herein, a “therapeutic agent” is a molecule or atom which is conjugated to an antibody moiety to produce a conjugate which is useful for therapy. Examples of therapeutic agents include drugs, toxins, immunomodulators, chelators, boron compounds, photoactive agents or dyes, and radioisotopes.

A “detectable label” is a molecule or atom which can be conjugated to an antibody moiety to produce a molecule useful for diagnosis. Examples of detectable labels include chelators, photoactive agents, radioisotopes, fluorescent agents, paramagnetic ions, or other marker moieties.

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075 (1985); Nilsson et al., Methods Enzymol. 198:3 (1991)), glutathione S transferase (Smith and Johnson, Gene 67:31 (1988)), Glu-Glu affinity tag (Grussenmeyer et al., Proc. Natl. Acad. Sci. USA 82:7952 (1985)), substance P, FLAG peptide (Hopp et al., Biotechnology 6:1204 (1988)), streptavidin binding peptide, or other antigenic epitope or binding domain. See, in general, Ford et al., Protein Expression and Purification 2:95 (1991). Nucleic acid molecules encoding affinity tags are available from commercial suppliers (e.g., Pharmacia Biotech, Piscataway, N.J.).

A “naked antibody” is an entire antibody, as opposed to an antibody fragment, which is not conjugated with a therapeutic agent. Naked antibodies include both polyclonal and monoclonal antibodies, as well as certain recombinant antibodies, such as chimeric and humanized antibodies.

As used herein, the term “antibody component” includes both an entire antibody and an antibody fragment.

An “immunoconjugate” is a conjugate of an antibody component with a therapeutic agent or a detectable label.

As used herein, the term “antibody fusion protein” refers to a recombinant molecule that comprises an antibody component and a therapeutic agent. Examples of therapeutic agents suitable for such fusion proteins include immunomodulators (“antibody-immunomodulator fusion protein”) and toxins (“antibody-toxin fusion protein”).

A “target polypeptide” or a “target peptide” is an amino acid sequence that comprises at least one epitope, and that is expressed on a target cell, such as a tumor cell, or a cell that carries an infectious agent antigen. T cells recognize peptide epitopes presented by a major histocompatibility complex molecule to a target polypeptide or target peptide and typically lyse the target cell or recruit other immune cells to the site of the target cell, thereby killing the target cell.

An “antigenic peptide” is a peptide which will bind a major histocompatibility complex molecule to form an MHC-peptide complex which is recognized by a T cell, thereby inducing a cytotoxic lymphocyte response upon presentation to the T cell. Thus, antigenic peptides are capable of binding to an appropriate major histocompatibility complex molecule and inducing a cytotoxic T cells response, such as cell lysis or specific cytokine release against the target cell which binds or expresses the antigen. The antigenic peptide can be bound in the context of a class I or class II major histocompatibility complex molecule, on an antigen presenting cell or on a target cell.

In eukaryotes, RNA polymerase II catalyzes the transcription of a structural gene to produce mRNA. A nucleic acid molecule can be designed to contain an RNA polymerase II template in which the RNA transcript has a sequence that is complementary to that of a specific mRNA. The RNA transcript is termed an “anti-sense RNA” and a nucleic acid molecule that encodes the anti-sense RNA is termed an “anti-sense gene.” Anti-sense RNA molecules are capable of binding to mRNA molecules, resulting in an inhibition of mRNA translation.

An “anti-sense oligonucleotide specific for pHHLA2” or an “pHHLA2 anti-sense oligonucleotide” is an oligonucleotide having a sequence (a) capable of forming a stable triplex with a portion of the pHHLA2 gene, or (b) capable of forming a stable duplex with a portion of an mRNA transcript of the pHHLA2 gene.

A “ribozyme” is a nucleic acid molecule that contains a catalytic center. The term includes RNA enzymes, self-splicing RNAs, self-cleaving RNAs, and nucleic acid molecules that perform these catalytic functions. A nucleic acid molecule that encodes a ribozyme is termed a “ribozyme gene.”

An “external guide sequence” is a nucleic acid molecule that directs the endogenous ribozyme, RNase P, to a particular species of intracellular mRNA, resulting in the cleavage of the mRNA by RNase P. A nucleic acid molecule that encodes an external guide sequence is termed an “external guide sequence gene.”

As used herein, an “antigen presenting cell” or “APC” is a cell that displays a foreign antigen complexed with MHC on its surface in a form that T cells can recognize it. The cells that can “present” antigen include B cells and cells of the monocyte lineage including dendritic cells, monocytes and macrophages.

The term “variant pHHLA2 gene” refers to nucleic acid molecules that encode a polypeptide having an amino acid sequence that is a modification of SEQ ID NO:2 or SEQ ID NO:5. Such variants include naturally-occurring polymorphisms of pHHLA2 genes, as well as synthetic genes that contain conservative amino acid substitutions of the amino acid sequence of SEQ ID NOs:2 or 5. Additional variant forms of pHHLA2 genes are nucleic acid molecules that contain insertions or deletions of the nucleotide sequences described herein. A variant pHHLA2 gene can be identified by determining whether the gene hybridizes with a nucleic acid molecule having the nucleotide sequence of SEQ ID NO:1 or SEQ ID NO:4, or its complement, under stringent conditions.

Alternatively, variant pHHLA2 genes can be identified by sequence comparison. Two amino acid sequences have “100% amino acid sequence identity” if the amino acid residues of the two amino acid sequences are the same when aligned for maximal correspondence. Similarly, two nucleotide sequences have “100% nucleotide sequence identity” if the nucleotide residues of the two nucleotide sequences are the same when aligned for maximal correspondence. Sequence comparisons can be performed using standard software programs such as those included in the LASERGENE bioinformatics computing suite, which is produced by DNASTAR (Madison, Wis.). Other methods for comparing two nucleotide or amino acid sequences by determining optimal alignment are well-known to those of skill in the art (see, for example, Peruski and Peruski, The Internet and the New Biology Tools for Genomic and Molecular Research (ASM Press, Inc. 1997), Wu et al. (eds.), “Information Superhighway and Computer Databases of Nucleic Acids and Proteins,” in Methods in Gene Biotechnology, pages 123-151 (CRC Press, Inc. 1997), and Bishop (ed.), Guide to Human Genome Computing, 2nd Edition (Academic Press, Inc. 1998)). Particular methods for determining sequence identity are described below.

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The term “ortholog” denotes a polypeptide or protein obtained from one species that is the functional counterpart of a polypeptide or protein from a different species. Sequence differences among orthologs are the result of speciation.

“Paralogs” are distinct but structurally related proteins made by an organism. Paralogs are believed to arise through gene duplication. For example, α-globin, β-globin, and myoglobin are paralogs of each other. Within the context of this invention, a “functional fragment” of a pHHLA2 gene refers to a nucleic acid molecule that encodes a portion of a pHHLA2 polypeptide which specifically binds with an anti-pHHLA2 antibody. For example, a functional fragment of a pHHLA2 gene described herein comprises a portion of the nucleotide sequence of SEQ ID NO:1 or SEQ ID NO:4, and encodes a polypeptide that specifically binds with an anti-pHHLA2 antibody.

As used herein, a polypeptide that “co-stimulates” a T cell is a polypeptide that, upon interaction with a cell-surface molecule on the T cell, enhances the response of the T cell. The T cell response that results from the interaction will be greater than the response in the absence of the polypeptide. The response of the T cell in the absence of the co-stimulatory polypeptide can be no response or it can be a response significantly lower than in the presence of the co-stimulatory polypeptide. It is understood that the response of the T cell can be an effector, helper, or suppressive response.

As used herein, an “activating stimulus” is a molecule that delivers an activating signal to a T cell, preferably through the antigen specific T cell receptor (TCR). The activating stimulus can be sufficient to elicit a detectable response in the T cell. Alternatively, the T cell may require co-stimulation (e.g., by a pHHLA2 polypeptide) in order to respond detectably to the activating stimulus. Examples of activating stimuli include, without limitation, antibodies that bind to the TCR or to a polypeptide of the CD3 complex that is physically associated with the TCR on the T cell surface, alloantigens, or an antigenic peptide bound to a MHC molecule.

As used herein, a “fragment” of a pHHLA2 polypeptide is a fragment of the polypeptide that is shorter than the full-length polypeptide, preferably shorter than the extracellular domain of pHHLA2. Generally, fragments will be five or more amino acids in length. An antigenic fragment has the ability to be recognized and bound by an antibody.

As used herein, a “functional fragment” of a pHHLA2 polypeptide is a fragment of the polypeptide that is shorter than the full-length polypeptide and has the ability to co-stimulate a T cell. In addition, a “functionally fragment” of the extracellular domain of pHHLA2 is shorter than the extracellular domain of the polypeptide and has the ability to antagonize the co-stimulatory activity of pHHLA2. Methods of establishing whether a fragment of an pHHLA2 molecule is functional are known in the art. For example, fragments of interest can be made by recombinant, synthetic, or proteolytic digestive methods. Such fragments can then be isolated and tested for their ability to co-stimulate T cells by procedures described herein.

Due to the imprecision of standard analytical methods, molecular weights and lengths of polymers are understood to be approximate values. When such a value is expressed as “about” X or “approximately” X, the stated value of X will be understood to be accurate to ±10%.

pHHLA2 ligand or co-receptor polypeptide is a polypeptide that is present on an antigen presenting cell, and which “co-stimulates” the T cell upon interaction with a cell-surface molecule on the T cell (counterpart co-receptor), and enhances the response of the T cell. The T cell response that results from the interaction will be greater than the response in the absence of the polypeptide. The response of the T cell in the absence of the co-stimulatory polypeptide can be no response or it can be a response significantly lower than in the presence of the co-stimulatory polypeptide. It is understood that the response of the T cell can be an effector, helper, or suppressive response.

The present invention provides an isolated receptor on antigen presenting cells APCs, which encodes a polypeptide having homology to the B7 family of proteins. The polypeptide has been designated pHHLA2. The nucleotide sequences of pHHLA2 are described in SEQ ID NO:1 (×1 variant) and SEQ ID NO:4 (×2 variant), and its deduced amino acid sequence is described in SEQ ID NO:2 and SEQ ID NO:5, respectively. The pHHLA2×1 polypeptide (SEQ ID NO:2) includes a signal sequence, comprising amino acid 1 (Met) to amino acid residue 22 (Gly) of SEQ ID NO:2, which is encoded by nucleotides 1-66 of SEQ ID NO:1. The mature polypeptide ranges from amino acid 23 (Ile) to amino acid 414 (Val) of SEQ ID NO:2, encoded by nucleotides 67-1242 of SEQ ID NO:1. The pHHLA2 polypeptide has an extracellular domain, a transmembrane domain and an intracellular domain. The extracellular domain of the mature polypeptide includes amino acid acid residues 23 (Ile) to 346 (Gly) of SEQ ID NO:2 (amino acid residues 1 (Met) to 313 (Gly) of SEQ ID NO:5), which is encoded by nucleotides 67-1038 of SEQ ID NO:1 (nucleotides 1-939 of SEQ ID NO:4). Within the extracellular domain of the mature polypeptide is the first of two immunoglobulin variable region (Igv1) between amino acid residues 39 (Val) and 139 (Gly) of SEQ ID NO:2 (amino acid residues 6 (Val) and 106 (Gly) of SEQ ID NO:5), which is encoded by nucleotides 115-417 of SEQ ID NO:1 (nucleotides 16-318 of SEQ ID NO:4). In addition, an immunoglobulin constant region (Igc) is also located in the extracellular domain of the mature polypeptide, which includes amino acid residues 236 (Ser) to 319 (Ile) of SEQ ID NO:2 (amino acid residues 203 (Ser) to 286 (Ile) of SEQ ID NO:5), which is encoded by nucleotides 706-957 of SEQ ID NO: 1 (nucleotides 607-858 of SEQ ID NO:4). The second immunoglobulin variable region (Igv2) is located between amino acid residues 230 (Gly) and 330 (His) of SEQ ID NO:2 (amino acid residues 197 (Gly) and 297 (His) of SEQ ID NO:5), which is encoded by nucleotides 688-990 of SEQ ID NO:1 (nucleotides 589-891 of SEQ ID NO:4). When referring to “pHHLA2”, pHHLA2 encompasses both pHHLA2×1 and pHHLA2×2.

The pHHLA2 mature polypeptide also includes a transmembrane domain which includes amino acid residues 347 (Leu) to 365 (Val) of SEQ ID NO:2 (amino acid residues 314 (Leu) to 332 (Val) of SEQ ID NO:5), which is encoded by nucleotides 1039-1095 of SEQ ID NO:1 (nucleotides 940-996 of SEQ ID NO:4).

The intracellular domain of the pHHLA2 mature polypeptide is located between amino acid residues 366 (Lys) and 414 (Val) of SEQ ID NO:2 (amino acid residues 333 (Lys) and 381 (Val) of SEQ ID NO:5), which is encoded by nucleotides 1096-1242 of SEQ ID NO:1 (nucleotides 997-1143 of SEQ ID NO:4).

Those skilled in the art will recognize that these domain boundaries are approximate, and are based on alignments with known proteins and predictions of protein folding.

The present invention provides polynucleotide molecules, including DNA and RNA molecules that encode the pHHLA2 polypeptides disclosed herein. Those skilled in the art will recognize that, in view of the degeneracy of the genetic code, considerable sequence variation is possible among these polynucleotide molecules. SEQ ID NOs:3 and 6 are degenerate DNA sequences that encompass all DNAs that encode the pHHLA2 polypeptide of SEQ ID NO:2 and SEQ ID NO:5, respectively, and fragments thereof. Those skilled in the art will recognize that the degenerate sequences of SEQ ID NOs:3 and 6 also provide all RNA sequences encoding SEQ ID NOs:2 and 5 by substituting U for T. Thus, pHHLA2 polynucleotides encoding pHHLA2 polypeptides of the present invention comprises nucleotide 1 to nucleotide 1242 of SEQ ID NO:3 and nucleotide 1 to nucleotide 1143 of SEQ ID NO:6 and their RNA equivalents are contemplated by the present invention. Table 1 sets forth the one-letter codes used within SEQ ID NOs:3 and 6 to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 1

| | | | |
|---|---|---|---|
| Nucleotide | Resolution | Complement | Resolution |

| | | | |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NOs:3 and 6 encompass all possible codons for a given amino acid, are set forth in Table 2.

TABLE 2

| | One | | |
|---|---|---|---|
| Amino | Letter | | Degenerate |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequences of SEQ ID NO:2 and SEQ ID NO:5. Variant sequences can be readily tested for functionality as described herein.

One of ordinary skill in the art will also appreciate that different species can exhibit “preferential codon usage.” In general, see, Grantham, et al., Nuc. Acids Res. 8:1893-912, 1980; Haas, et al. Curr. Biol. 6:315-24, 1996; Wain-Hobson et al., Gene 13:355-64, 1981; Grosjean and Fiers, Gene 18:199-209, 1982; Holm, Nuc. Acids Res. 14:3075-87, 1986; Ikemura, J. Mol. Biol. 158:573-97, 1982. As used herein, the term “preferential codon usage” or “preferential codons” is a term of art referring to protein translation codons that are most frequently used in cells of a certain species, thus favoring one or a few representatives of the possible codons encoding each amino acid (See Table 2). For example, the amino acid Threonine (Thr) may be encoded by ACA, ACC, ACG, or ACT, but in mammalian cells ACC is the most commonly used codon; in other species, for example, insect cells, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequences disclosed in SEQ ID NO:3 and SEQ ID NO:6 serve as templates for optimizing expression of pHHLA2 polynucleotides in various cell types and species commonly used in the art and disclosed herein. Sequences containing preferential codons can be tested and optimized for expression in various species, and tested for functionality as disclosed herein.

As previously noted, the isolated polynucleotides of the present invention include DNA and RNA. Methods for preparing DNA and RNA are well known in the art. In general, RNA is isolated from a tissue or cell that produces large amounts of pHHLA2 RNA. Such tissues and cells are identified by Northern blotting (Thomas, Proc. Natl. Acad. Sci. USA 77:5201, 1980), and include PBLs, spleen, thymus, bone marrow, prostate, and lymph tissues, human erythroleukemia cell lines, acute monocytic leukemia cell lines, other lymphoid and hematopoietic cell lines, and the like. Total RNA can be prepared using guanidinium isothiocyanate extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-12, 1972). Complementary DNA (cDNA) is prepared from poly(A)⁺ RNA using known methods. In the alternative, genomic DNA can be isolated. Polynucleotides encoding pHHLA2 polypeptides are then identified and isolated by, for example, hybridization or polymerase chain reaction (PCR) (Mullis, U.S. Pat. No. 4,683,202).

A full-length clone encoding pHHLA2 can be obtained by conventional cloning procedures. Complementary DNA (cDNA) clones are preferred, although for some applications (e.g., expression in transgenic animals) it may be preferable to use a genomic clone, or to modify a cDNA clone to include at least one genomic intron. Methods for preparing cDNA and genomic clones are well known and within the level of ordinary skill in the art, and include the use of the sequence disclosed herein, or parts thereof, for probing or priming a library. Expression libraries can be probed with antibodies to pHHLA2, receptor fragments, or other specific binding partners.

The polynucleotides of the present invention can also be synthesized using DNA synthesis machines. Currently the method of choice is the phosphoramidite method. If chemically synthesized double stranded DNA is required for an application such as the synthesis of a gene or a gene fragment, then each complementary strand is made separately. The production of short polynucleotides (60 to 80 bp) is technically straightforward and can be accomplished by synthesizing the complementary strands and then annealing them. However, for producing longer polynucleotides (>300 bp), special strategies are usually employed, because the coupling efficiency of each cycle during chemical DNA synthesis is seldom 100%. To overcome this problem, synthetic genes (double-stranded) are assembled in modular form from single-stranded fragments that are from 20 to 100 nucleotides in length.

An alternative way to prepare a full-length gene is to synthesize a specified set of overlapping oligonucleotides (40 to 100 nucleotides). After the 3′ and 5′ short overlapping complementary regions (6 to 10 nucleotides) are annealed, large gaps still remain, but the short base-paired regions are both long enough and stable enough to hold the structure together. The gaps are filled and the DNA duplex is completed via enzymatic DNA synthesis by E. coli DNA polymerase 1. After the enzymatic synthesis is completed, the nicks are sealed with T4 DNA ligase. Double-stranded constructs are sequentially linked to one another to form the entire gene sequence which is verified by DNA sequence analysis. See Glick and Pasternak, Molecular Biotechnology, Principles & Applications of Recombinant DNA, (ASM Press, Washington, D.C. 1994); Itakura et al., Annu. Rev. Biochem. 53: 323-56, 1984 and Climie et al., Proc. Natl. Acad. Sci. USA 87:633-7, 1990. Moreover, other sequences are generally added that contain signals for proper initiation and termination of transcription and translation.

The present invention also provides reagents which will find use in diagnostic applications. For example, the pHHLA2 gene, a probe comprising pHHLA2 DNA or RNA or a subsequence thereof, can be used to determine if the pHHLA2 gene is present on a human chromosome, such as chromosome 3, or if a gene mutation has occurred. pHHLA2 is located at the q13.13 region of chromosome 3. Detectable chromosomal aberrations at the pHHLA2 gene locus include, but are not limited to, aneuploidy, gene copy number changes, loss of heterozygosity (LOH), translocations, insertions, deletions, restriction site changes and rearrangements. Such aberrations can be detected using polynucleotides of the present invention by employing molecular genetic techniques, such as restriction fragment length polymorphism (RFLP) analysis, short tandem repeat (STR) analysis employing PCR techniques, and other genetic linkage analysis techniques known in the art (Sambrook et al., ibid.; Ausubel et. al., ibid.; Marian, Chest 108:255-65, 1995).

The precise knowledge of a gene's position can be useful for a number of purposes, including: 1) determining if a sequence is part of an existing contig and obtaining additional surrounding genetic sequences in various forms, such as YACs, BACs or cDNA clones; 2) providing a possible candidate gene for an inheritable disease which shows linkage to the same chromosomal region; and 3) cross-referencing model organisms, such as mouse, which may aid in determining what function a particular gene might have.

A diagnostic could assist physicians in determining the type of disease and appropriate associated therapy, or assistance in genetic counseling. As such, the inventive anti-pHHLA2 antibodies, polynucleotides, and polypeptides can be used for the detection of pHHLA2 polypeptide, mRNA or anti-pHHLA2 antibodies, thus serving as markers and be directly used for detecting or genetic diseases or cancers, as described herein, using methods known in the art and described herein. Further, pHHLA2 polynucleotide probes can be used to detect abnormalities or genotypes associated with chromosome 3q13.13 deletions and translocations associated with human diseases, or other translocations involved with malignant progression of tumors or other 3q13.13 mutations, which are expected to be involved in chromosome rearrangements in malignancy; or in other cancers. Similarly, pHHLA2 polynucleotide probes can be used to detect abnormalities or genotypes associated with chromosome 3 trisomy and chromosome loss associated with human diseases or spontaneous abortion. Thus, pHHLA2 polynucleotide probes can be used to detect abnormalities or genotypes associated with these defects.

In general, the diagnostic methods used in genetic linkage analysis, to detect a genetic abnormality or aberration in a patient, are known in the art. Analytical probes will be generally at least 20 nt in length, although somewhat shorter probes can be used (e.g., 14-17 nt). PCR primers are at least 5 nt in length, preferably 15 or more, more preferably 20-30 nt. For gross analysis of genes, or chromosomal DNA, a pHHLA2 polynucleotide probe may comprise an entire exon or more. In general, the diagnostic methods used in genetic linkage analysis, to detect a genetic abnormality or aberration in a patient, are known in the art. Most diagnostic methods comprise the steps of (a) obtaining a genetic sample from a potentially diseased patient, diseased patient or potential non-diseased carrier of a recessive disease allele; (b) producing a first reaction product by incubating the genetic sample with a pHHLA2 polynucleotide probe wherein the polynucleotide will hybridize to complementary polynucleotide sequence, such as in RFLP analysis or by incubating the genetic sample with sense and antisense primers in a PCR reaction under appropriate PCR reaction conditions; (iii) visualizing the first reaction product by gel electrophoresis and/or other known methods such as visualizing the first reaction product with a pHHLA2 polynucleotide probe wherein the polynucleotide will hybridize to the complementary polynucleotide sequence of the first reaction; and (iv) comparing the visualized first reaction product to a second control reaction product of a genetic sample from wild type patient, or a normal or control individual. A difference between the first reaction product and the control reaction product is indicative of a genetic abnormality in the diseased or potentially diseased patient, or the presence of a heterozygous recessive carrier phenotype for a non-diseased patient, or the presence of a genetic defect in a tumor from a diseased patient, or the presence of a genetic abnormality in a fetus or pre-implantation embryo. For example, a difference in restriction fragment pattern, length of PCR products, length of repetitive sequences at the pHHLA2 genetic locus, and the like, are indicative of a genetic abnormality, genetic aberration, or allelic difference in comparison to the normal wild type control. Controls can be from unaffected family members, or unrelated individuals, depending on the test and availability of samples. Genetic samples for use within the present invention include genomic DNA, mRNA, and cDNA isolated from any tissue or other biological sample from a patient, which includes, but is not limited to, blood, saliva, semen, embryonic cells, amniotic fluid, and the like. The polynucleotide probe or primer can be RNA or DNA, and will comprise a portion of SEQ ID NO:1, the complement of SEQ ID NO:1, or an RNA equivalent thereof. Such methods of showing genetic linkage analysis to human disease phenotypes are well known in the art. For reference to PCR based methods in diagnostics see generally, Mathew (ed.), Protocols in Human Molecular Genetics (Humana Press, Inc. 1991), White (ed.), PCR Protocols: Current Methods and Applications (Humana Press, Inc. 1993), Cotter (ed.), Molecular Diagnosis of Cancer (Humana Press, Inc. 1996), Hanausek and Walaszek (eds.), Tumor Marker Protocols (Humana Press, Inc. 1998), Lo (ed.), Clinical Applications of PCR (Humana Press, Inc. 1998), and Meltzer (ed.), PCR in Bioanalysis (Humana Press, Inc. 1998).

Mutations associated with the pHHLA2 locus can be detected using nucleic acid molecules of the present invention by employing standard methods for direct mutation analysis, such as restriction fragment length polymorphism analysis, short tandem repeat analysis employing PCR techniques, amplification-refractory mutation system analysis, single-strand conformation polymorphism detection, RNase cleavage methods, denaturing gradient gel electrophoresis, fluorescence-assisted mismatch analysis, and other genetic analysis techniques known in the art (see, for example, Mathew (ed.), Protocols in Human Molecular Genetics (Humana Press, Inc. 1991), Marian, Chest 108:255 (1995), Coleman and Tsongalis, Molecular Diagnostics (Human Press, Inc. 1996), Elles (ed.) Molecular Diagnosis of Genetic Diseases (Humana Press, Inc. 1996), Landegren (ed.), Laboratory Protocols for Mutation Detection (Oxford University Press 1996), Birren et al. (eds.), Genome Analysis, Vol. 2: Detecting Genes (Cold Spring Harbor Laboratory Press 1998), Dracopoli et al. (eds.), Current Protocols in Human Genetics (John Wiley & Sons 1998), and Richards and Ward, “Molecular Diagnostic Testing,” in Principles of Molecular Medicine, pages 83-88 (Humana Press, Inc. 1998). Direct analysis of a pHHLA2 gene for a mutation can be performed using a subject's genomic DNA. Methods for amplifying genomic DNA, obtained for example from peripheral blood lymphocytes, are well-known to those of skill in the art (see, for example, Dracopoli et al. (eds.), Current Protocols in Human Genetics, at pages 7.1.6 to 7.1.7 (John Wiley & Sons 1998)).

The present invention further provides counterpart polypeptides and polynucleotides from other species (orthologs). These species include, but are not limited to mammalian, avian, amphibian, reptile, fish, insect and other vertebrate and invertebrate species. Of particular interest are pHHLA2 polypeptides from other mammalian species, including murine, porcine, ovine, bovine, canine, feline, equine, and other primate polypeptides. Orthologs of human pHHLA2 can be cloned using information and compositions provided by the present invention in combination with conventional cloning techniques. For example, a cDNA can be cloned using mRNA obtained from a tissue or cell type that expresses pHHLA2 as disclosed herein. Suitable sources of mRNA can be identified by probing Northern blots with probes designed from the sequences disclosed herein. A library is then prepared from mRNA of a positive tissue or cell line. A pHHLA2-encoding cDNA can then be isolated by a variety of methods, such as by probing with a complete or partial human cDNA or with one or more sets of degenerate probes based on the disclosed sequences. A cDNA can also be cloned using PCR (Mullis, supra.), using primers designed from the representative human pHHLA2 sequence disclosed herein. Within an additional method, the cDNA library can be used to transform or transfect host cells, and expression of the cDNA of interest can be detected with an antibody to pHHLA2 polypeptide. Similar techniques can also be applied to the isolation of genomic clones.

The present invention also provides isolated pHHLA2 polypeptides that are substantially similar to the polypeptides of SEQ ID NO:2 or SEQ ID NO:5. The term “substantially similar” is used herein to denote polypeptides having at least 50%, at least 60%, at least 70%, at least 80%, at least 90%, at least 91%, at least 92%, at least 93%, at least 94%, at least 95%, at least 96%, at least 97%, at least 98%, at least 99%, at least 99.5%, or greater than 99.5% sequence identity to the sequences shown in SEQ ID NO:2 or SEQ ID NO:5. Percent sequence identity is determined by conventional methods. See, for example, Altschul et al., Bull. Math. Bio. 48: 603-616, 1986 and Henikoff and Henikoff, Proc. Natl. Acad. Sci. USA 89:10915-10919, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown in Table 3 (amino acids are indicated by the standard one-letter codes). The percent identity is then calculated as:

$$\frac{Total number of identical matches}{[ \begin{matrix}length of the longer sequence plus the \\ number of gaps introduced into the longer \\ sequence in order to align the two sequences\end{matrix} ]} \times 100$$

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Sequence identity of polynucleotide molecules is determined by similar methods using a ratio as disclosed above.

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant pHHLA2. The FASTA algorithm is described by Pearson and Lipman, Proc. Nat'l Acad. Sci. USA 85:2444 (1988), and by Pearson, Meth. Enzymol. 183:63 (1990).

Briefly, FASTA first characterizes sequence similarity by identifying regions shared by the query sequence (e.g., SEQ ID NO:2 and SEQ ID NO:5) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Preferred parameters for FASTA analysis are: ktup=1, gap opening penalty=10, gap extension penalty=1, and substitution matrix=BLOSUM62, with other parameters set as default. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from three to six, most preferably three, with other FASTA program parameters set as default.

The BLOSUM62 table (Table 3) is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed below), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Within certain embodiments of the invention, the isolated nucleic acid molecules can hybridize under stringent conditions to nucleic acid molecules comprising nucleotide sequences disclosed herein. For example, such nucleic acid molecules can hybridize under stringent conditions to nucleic acid molecules comprising the nucleotide sequence of SEQ ID NO:1 or SEQ ID NO:4, to nucleic acid molecules consisting of the nucleotide sequence of SEQ ID NO:1 or SEQ ID NO:4, or to nucleic acid molecules consisting of a nucleotide sequence complementary to SEQ ID NO:1 or SEQ ID NO:4. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe.

A pair of nucleic acid molecules, such as DNA-DNA, RNA-RNA and DNA-RNA, can hybridize if the nucleotide sequences have some degree of complementarity. Hybrids can tolerate mismatched base pairs in the double helix, but the stability of the hybrid is influenced by the degree of mismatch. The Tₘ of the mismatched hybrid decreases by 1° C. for every 1-1.5% base pair mismatch. Varying the stringency of the hybridization conditions allows control over the degree of mismatch that will be present in the hybrid. The degree of stringency increases as the hybridization temperature increases and the ionic strength of the hybridization buffer decreases. Stringent hybridization conditions encompass temperatures of about 5-25° C. below the Tₘ of the hybrid and a hybridization buffer having up to 1 M Na⁺. Higher degrees of stringency at lower temperatures can be achieved with the addition of formamide which reduces the Tₘ of the hybrid about 1° C. for each 1% formamide in the buffer solution. Generally, such stringent conditions include temperatures of 20-70° C. and a hybridization buffer containing up to 6×SSC and 0-50% formamide. A higher degree of stringency can be achieved at temperatures of from 40-70° C. with a hybridization buffer having up to 4×SSC and from 0-50% formamide. Highly stringent conditions typically encompass temperatures of 42-70° C. with a hybridization buffer having up to 1×SSC and 0-50% formamide. Different degrees of stringency can be used during hybridization and washing to achieve maximum specific binding to the target sequence. Typically, the washes following hybridization are performed at increasing degrees of stringency to remove non-hybridized polynucleotide probes from hybridized complexes.

The above conditions are meant to serve as a guide and it is well within the abilities of one skilled in the art to adapt these conditions for use with a particular polypeptide hybrid. The Tₘ for a specific target sequence is the temperature (under defined conditions) at which 50% of the target sequence will hybridize to a perfectly matched probe sequence. Those conditions which influence the Tₘ include, the size and base pair content of the polynucleotide probe, the ionic strength of the hybridization solution, and the presence of destabilizing agents in the hybridization solution. Numerous equations for calculating Tₘ are known in the art, and are specific for DNA, RNA and DNA-RNA hybrids and polynucleotide probe sequences of varying length (see, for example, Sambrook et al., Molecular Cloning: A Laboratory Manual, Second Edition (Cold Spring Harbor Press 1989); Ausubel et al., (eds.), Current Protocols in Molecular Biology (John Wiley and Sons, Inc. 1987); Berger and Kimmel (eds.), Guide to Molecular Cloning Techniques, (Academic Press, Inc. 1987); and Wetmur, Crit. Rev. Biochem. Mol. Biol. 26:227 (1990)). Sequence analysis software, as well as sites on the Internet, are available tools for analyzing a given sequence and calculating Tₘ based on user defined criteria. Such programs can also analyze a given sequence under defined conditions and identify suitable probe sequences. Typically, hybridization of longer polynucleotide sequences, >50 base pairs, is performed at temperatures of about 20-25° C. below the calculated Tₘ. For smaller probes, <50 base pairs, hybridization is typically carried out at the Tₘ or 5-10° C. below. This allows for the maximum rate of hybridization for DNA-DNA and DNA-RNA hybrids.

The length of the polynucleotide sequence influences the rate and stability of hybrid formation. Smaller probe sequences, <50 base pairs, reach equilibrium with complementary sequences rapidly, but may form less stable hybrids. Incubation times of anywhere from minutes to hours can be used to achieve hybrid formation. Longer probe sequences come to equilibrium more slowly, but form more stable complexes even at lower temperatures. Incubations are allowed to proceed overnight or longer. Generally, incubations are carried out for a period equal to three times the calculated Cot time. Cot time, the time it takes for the polynucleotide sequences to reassociate, can be calculated for a particular sequence by methods known in the art.

The base pair composition of polynucleotide sequence will effect the thermal stability of the hybrid complex, thereby influencing the choice of hybridization temperature and the ionic strength of the hybridization buffer. A-T pairs are less stable than G-C pairs in aqueous solutions containing sodium chloride. Therefore, the higher the G-C content, the more stable the hybrid. Even distribution of G and C residues within the sequence also contribute positively to hybrid stability. In addition, the base pair composition can be manipulated to alter the Tₘ of a given sequence. For example, 5-methyldeoxycytidine can be substituted for deoxycytidine and 5-bromodeoxyuridine can be substituted for thymidine to increase the Tₘ, whereas 7-deazz-2′-deoxyguanosine can be substituted for guanosine to reduce dependence on Tₘ.

The ionic concentration of the hybridization buffer also affects the stability of the hybrid. Hybridization buffers generally contain blocking agents such as Denhardt's solution (Sigma Chemical Co., St. Louis, Mo.), denatured salmon sperm DNA, tRNA, milk powders (BLOTTO), heparin or SDS, and a Na⁺ source, such as SSC (1×SSC: 0.15 M sodium chloride, 15 mM sodium citrate) or SSPE (1×SSPE: 1.8 M NaCl, 10 mM NaH₂PO₄, 1 mM EDTA, pH 7.7). By decreasing the ionic concentration of the buffer, the stability of the hybrid is increased. Typically, hybridization buffers contain from between 10 mM-1 M Na⁺. The addition of destabilizing or denaturing agents such as formamide, tetralkylammonium salts, guanidinium cations or thiocyanate cations to the hybridization solution will alter the Tₘ of a hybrid. Typically, formamide is used at a concentration of up to 50% to allow incubations to be carried out at more convenient and lower temperatures. Formamide also acts to reduce non-specific background when using RNA probes.

As an illustration, a nucleic acid molecule encoding a variant pHHLA2 polypeptide can be hybridized with a nucleic acid molecule having the nucleotide sequence of SEQ ID NO: 1 (or its complement) at 42° C. overnight in a solution comprising 50% formamide, 5×SSC (1×SSC: 0.15 M sodium chloride and 15 mM sodium citrate), 50 mM sodium phosphate (pH 7.6), 5×Denhardt's solution (100×Denhardt's solution: 2% (w/v) Ficoll 400, 2% (w/v) polyvinylpyrrolidone, and 2% (w/v) bovine serum albumin, 10% dextran sulfate, and 20 μg/ml denatured, sheared salmon sperm DNA. One of skill in the art can devise variations of these hybridization conditions. For example, the hybridization mixture can be incubated at a higher temperature, such as about 65° C., in a solution that does not contain formamide. Moreover, premixed hybridization solutions are available (e.g. EXPRESSHYB Hybridization Solution from CLONTECH Laboratories, Inc.), and hybridization can be performed according to the manufacturer's instructions.

Following hybridization, the nucleic acid molecules can be washed to remove non-hybridized nucleic acid molecules under stringent conditions, or under highly stringent conditions. Typical stringent washing conditions include washing in a solution of 0.5×-2×SSC with 0.1% sodium dodecyl sulfate (SDS) at 55-65° C. That is, nucleic acid molecules encoding a variant zacrp8 polypeptide remained hybridized following stringent washing conditions with a nucleic acid molecule having the nucleotide sequence of SEQ ID NO: 1 (or its complement), in which the wash stringency is equivalent to 0.5×-2×SSC with 0.1% SDS at 55-65° C., including 0.5×SSC with 0.1% SDS at 55° C., or 2×SSC with 0.1% SDS at 65° C. One of skill in the art can readily devise equivalent conditions, for example, by substituting the SSPE for SSC in the wash solution.

Typical highly stringent washing conditions include washing in a solution of 0.1×-0.2×SSC with 0.1% sodium dodecyl sulfate (SDS) at 50-65° C. In other words, nucleic acid molecules encoding a variant pHHLA2 polypeptide remained hybridized following stringent washing conditions with a nucleic acid molecule having the nucleotide sequence of SEQ ID NO:1 (or its complement), in which the wash stringency is equivalent to 0.1×-0.2×SSC with 0.1% SDS at 50-65° C., including 0.1×SSC with 0.1% SDS at 50° C., or 0.2×SSC with 0.1% SDS at 65° C.

Variant pHHLA2 polypeptides or substantially homologous pHHLA2 polypeptides are characterized as having one or more amino acid substitutions, deletions or additions. These changes are preferably of a minor nature, that is conservative amino acid substitutions (see Table 4) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag. The present invention thus includes polypeptides that comprise a sequence that is at least 70%, at least 80%, at least 90%, at least 91%, at least 92%, at least 93%, at least 94%, at least 95%, at least 96%, at least 97%, at least 98%, at least 99%, at least 99.5%, or greater than 99.5% to the corresponding region of SEQ ID NO:2 or SEQ ID NO:5 excluding the tags, extension, linker sequences and the like. Polypeptides comprising affinity tags can further comprise a proteolytic cleavage site between the pHHLA2 polypeptide and the affinity tag. Suitable sites include thrombin cleavage sites and factor Xa cleavage sites.

TABLE 4
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

A full-length pHHLA2 polypeptide expressed on an antigen presenting cell has been shown (see Example 5) to co-stimulate T cells. It is well established that activated T cells secrete a number of inflammatory cytokines, e.g., IFNγ, TNFα, IL-1β, IL-2, IL-6, IL-12, IL-13, IL-17, IL-18, IL-21 and IL-23. Many of these cytokines have been shown to be over-expressed, for example, in human IBD samples and are therefore implicated in the initiation and perpetuation of the pro-inflammatory immune response in the gut. Accordingly, the inhibition of pHHLA2's co-stimulation of T cells by a soluble form of pHHLA2 or a pHHLA2 antibody or fragment thereof would be beneficial to those patients suffering from gut (see tissue expression data in Example 6) inflammatory diseases with an immunological component, such as Crohn's disease, ulcerative colitis, celiac disease, graft-versus-host disease, and irritable bowel syndrome. The soluble pHHLA2 polypeptide (e.g., amino acid residues 23-346 of SEQ ID NO:2 or amino acid residues 1-313 of SEQ ID NO:5); fusion proteins of same fused inframe or conjugated to an immunoglobulin heavy chain constant region (e.g., Fc2), such as isotypes IgG (i.e., IgG1, IgG2, IgG3, or IgG4), IgM, IgD, IgA (IgA1 or IgA2) or IgE, or conjugated to a polyalkyl oxide moiety, such as polyethylene glycol, optionally branched or linear with molecular weights of 5 kD-60 kD; and antibodies and antibody fragments would inhibit endogenous pHHLA2 on the APC from binding its T cell counterpart receptor and co-stimulating the T cell.

Another embodiment of the present invention is an isolated soluble pHHLA2 polypeptide comprising a sequence of amino acid residues that is has at least 95% sequence identity with amino acid residues 23-346 of SEQ ID NO:2 or amino acid residues 1-313 of SEQ ID NO:5, wherein the polypeptide inhibits the costimulation of T cells. The polypeptide may be amino acid residues 23-346 of SEQ ID NO:2 or amino acid residues 1-313 of SEQ ID NO:5.

Another embodiment of the present invention an isolated polynucleotide encoding a soluble polypeptide wherein the encoded polypeptide comprises a sequence of amino acid residues that is has at least 95% sequence identity with amino acid residues 23-346 of SEQ ID NO:2 or amino acid residues 1-313 of SEQ ID NO:5, wherein the encoded polypeptide inhibits the costimulation T cells. The isolated polynucleotide may be nucleotides 67-1038 of SEQ ID NO:1 or nucleotides 1-939 of SEQ ID NO:4.

Another embodiment of the present invention an isolated polynucleotide comprising nucleotides selected from the group consisting of 67-1038 of SEQ ID NO:1, 1-1038 of SEQ ID NO:1, 67-1095 of SEQ ID NO:1, 1-1095 of SEQ ID NO:1, 67-1242 of SEQ ID NO:1, 1-1242 of SEQ ID NO:1, 1-939 of SEQ ID NO:4, 1-996 of SEQ ID NO:4, and 1-1143 of SEQ ID NO:4. Optionally, an isolated polynucleotide that hybridizes 67-1038 of SEQ ID NO:1, 1-1038 of SEQ ID NO:1, 67-1095 of SEQ ID NO:1, 1-1095 of SEQ ID NO:1, 67-1242 of SEQ ID NO:1, 1-1242 of SEQ ID NO:1, 1-939 of SEQ ID NO:4, 1-996 of SEQ ID NO:4, and 1-1143 of SEQ ID NO:4 under stringent conditions of hybridization in buffer containing 5×SSC, 5×Denhardt's, 0.5% SDS, 1 mg salmon sperm/25 mls of hybridization solution incubated at 65° C. overnight, followed by high stringency washing with 0.2×SSC/0.1% SDS at 65° C., wherein the isolated polynucleotide encodes a soluble polypeptide that inhibits the costimulation T cells.

Another embodiment of the present invention is an expression vector comprising the following operably linked elements: a transcription promoter; a DNA segment encoding a polypeptide comprising a sequence of amino acid residues that is has at least 95% sequence identity with amino acid residues 23-346 of SEQ ID NO:2 or amino acid residues 1-313 of SEQ ID NO:5, wherein the polypeptide inhibits the costimulation of T cells; and a transcription terminator. The encoded polypeptide may be amino acid residues 23-346 of SEQ ID NO:2 or amino acid residues 1-313 of SEQ ID NO:5. Another embodiment of the present invention is a cultured cell into which has been introduced the expression vector, wherein the cell expresses the polypeptide encoded by the DNA segment.

Another embodiment of the present invention is a method of producing a polypeptide comprising culturing a cell into which has been introduced an expression vector as described herein, wherein the cell expresses the polypeptide encoded by the DNA segment; and recovering the expressed polypeptide.

Another embodiment of the present invention is an isolated or purified antibody or antibody fragment that specifically binds to a polypeptide comprising or consisting of a sequence of amino acid residues that is has at least 95% sequence identity with amino acid residues 23-346 of SEQ ID NO:2 or amino acid residues 1-313 of SEQ ID NO:5. The polypeptide may be amino acid residues 23-346 of SEQ ID NO:2 or amino acid residues 1-313 of SEQ ID NO:5. The isolated or purified antibody or antibody fragment may selectively bind to an epitope in the extracellular domain of pHHLA2. The isolated or purified antibody or antibody fragment may bind to the extracellular domain of pHHLA2 and inhibit the binding of pHHLA2 to its T-cell counter-receptor. The isolated or purified antibody may be a polyclonal antibody, a murine monoclonal antibody, a humanized antibody derived from a murine monoclonal antibody, an antibody fragment, neutralizing antibody, and a human monoclonal antibody. The isolated or purified antibody fragment may be a F(ab′), F(ab), F(ab′)₂, Fab′, Fab, Fv, scFv, and minimal recognition unit.

Another embodiment of the present invention is an anti-idiotype antibody comprising an anti-idiotype antibody that specifically binds to an antibody or antibody fragment as described herein.

Another embodiment of the present invention is a fusion protein comprising a polypeptide comprising a sequence of amino acid residues that has at least 95% sequence identity with amino acid residues 23-346 of SEQ ID NO:2 or amino acid residues 1-313 of SEQ ID NO:5; and a polyalkyl oxide moiety, wherein the fusion protein inhibits the co-stimulation of T cells. The polypeptide may be amino acid residues 23-346 of SEQ ID NO:2 or amino acid residues 1-313 of SEQ ID NO:5. The polyalkyl oxide moiety may be polyethylene glycol (PEG). The PEG may be N-terminally or C-terminally conjugated to the polypeptide and may comprise, for instance, a 20 kD or 30 kD monomethoxy-PEG propionaldehyde. The PEG may be linear or branched. The administration of fusion protein to a patient may inhibit the costimulation of T cells by binding to pHHLA2's T cell counter-receptor and thus inhibiting endoenous pHHLA2, expressed on antigen presenting cells, from binding to its T cell counter-receptor and activating the T cell.

Another embodiment of the present invention is a fusion protein comprising a polypeptide comprising a sequence of amino acid residues that has at least 95% sequence identity with amino acid residues 23-346 of SEQ ID NO:2 or amino acid residues 1-313 of SEQ ID NO:5; and an immunoglobulin heavy chain constant region, wherein the fusion protein inhibits the co-stimulation of T cells. The polypeptide may be amino acid residues 23-346 of SEQ ID NO:2 or amino acid residues 1-313 of SEQ ID NO:5. The immunoglobulin heavy chain constant region may be an Fc fragment. The immunoglobulin heavy chain constant region may be an isotype selected from the group consisting of an IgG, IgM, IgE, IgA and IgD. The IgG isotype may be IgG1, IgG2, IgG3, or IgG4.

Another embodiment of the present invention is a formulation comprising: an isolated soluble polypeptide comprising a sequence of amino acid residues that is has at least 95% sequence identity with amino acid residues 23-346 of SEQ ID NO:2 or amino acid residues 1-313 of SEQ ID NO:5; and pharmaceutically acceptable vehicle. The isolated soluble polypeptide may be amino acid residues 23-346 of SEQ ID NO:2 or amino acid residues 1-313 of SEQ ID NO:5. The formulation may be packaged in a kit.

Another embodiment of the present invention is a formulation comprising: an antibody or antibody fragment as described herein; and pharmaceutically acceptable vehicle. The formulation may be packaged in a kit.

Another embodiment of the present invention is a method of inhibiting the co-stimulation a T cell, the method comprising contacting the T cell with a soluble polypeptide, the sequence of which comprises a sequence having at least 95% identify with amino acid residues 23-346 of SEQ ID NO:2 or amino acid residues 1-313 of SEQ ID NO:5, wherein the polypeptide inhibits the co-stimulation of the T cell. The soluble polypeptide may be amino acid residues 23-346 of SEQ ID NO:2 or amino acid residues 1-313 of SEQ ID NO:5. The contacting may comprise culturing the polypeptide with the T cell in vitro. The T cell may be in a patient. The contacting may comprise administering the polypeptide to the patient. The contacting may comprise administering a nucleic acid encoding the polypeptide to the patient. The method may further comprise (a) providing a recombinant cell which is the progeny of a cell obtained from the patient and has been transfected or transformed ex vivo with a nucleic acid molecule encoding the polypeptide so that the cell expresses the polypeptide; and (b) administering the cell to the patient. The recombinant cell may be an antigen presenting cell (APC) and expresses the polypeptide on its surface. The method may include that prior to the administering, the APC is pulsed with an antigen or an antigenic peptide. The patient may be suffering from an inflammatory disease selected from the group consisting of Crohn's disease, ulcerative colitis, graft versus host disease, celiac disease, and irritable bowel syndrome.

Another embodiment of the present invention is a method of treating, preventing, inhibiting the progression of, delaying the onset of and/or reducing at least one of the symptoms or conditions associated with a disease selected from the group consisting of Crohn's disease, ulcerative colitis, celiac disease, Graft-versus-host disease, and irritable bowel syndrome comprising administering to the patient an effective amount of a formulation as described herein.

The present invention provides an isolated pHHLA2 soluble polypeptide, the amino acid sequence of which comprises a sequence having at least 95% sequence identity with amino acid residues 23-346 of SEQ ID NO:2, wherein the isolated pHHLA2 polypeptide inhibits the costimulation of T cells. The isolated pHHLA2 polypeptide may comprise amino acid residues 23-414 of SEQ ID NO:2. The isolated pHHLA2 polypeptide can be a soluble pHHLA2 polypeptide. The soluble pHHLA2 may be fused to another protein. The other protein may be a constant region of an antibody, e.g., Fc2, polyethylene glycol or serum albumin.

The present invention provides an isolated soluble pHHLA2 polypeptide, the amino acid sequence of which comprises a sequence having at least 95% sequence identity with amino acid residues amino acid residues 1-313 of SEQ ID NO:5, wherein the isolated soluble pHHLA2 polypeptide inhibits the costimulation T cells. The isolated pHHLA2 polypeptide can be a soluble pHHLA2 co-receptor. The isolated pHHLA2 co-receptor may comprise amino acid residues 1-381 of SEQ ID NO:5. The isolated pHHLA2 co-receptor may comprise amino acid residues 1-313 of SEQ ID NO:5. The isolated pHHLA2 polypeptide can be a soluble pHHLA2 polypeptide. The soluble pHHLA2 may be fused to another protein. The other protein may be a constant region of an antibody, e.g., Fc2, polyethylene glycol or serum albumin.

The present invention also provides an isolated polynucleotide comprising a sequence that encodes a polypeptide the amino acid sequence of which having at least 95 percent sequence identity with amino acid residues 23-346 of SEQ ID NO:2, wherein the polypeptide inhibits the costimulation of a T cell. The polynucleotide may optionally encode a polypeptide comprising amino acid residues 23-414 of SEQ ID NO:2 or 23-346 of SEQ ID NO:2. The encoded polypeptide may be soluble. The polynucleotide may comprise nucleotides 67-1038 of SEQ ID NO:1.

The present invention also provides an isolated polynucleotide comprising a sequence that encodes a polypeptide the amino acid sequence of which having at least 95 percent sequence identity with amino acid residues 1-313 of SEQ ID NO:5, wherein the polypeptide inhibits the costimulation of a T cell. The polynucleotide may optionally encode a polypeptide comprising amino acid residues 1-381 of SEQ ID NO:5 or 1-313 of SEQ ID NO:5. The encoded polypeptide may be soluble. The polynucleotide may comprises nucleotides 1-939 of SEQ ID NO:4.

The present invention further provides a variety of other polypeptide fusions and related multimeric (e.g., homodimeric or heterodimeric) proteins comprising one or more polypeptide fusions. For example, a soluble pHHLA2 polypeptide (the extracellular domain of pHHLA2 or fragment thereof, e.g., amino acid residues 23-346 of SEQ ID NO:2 and amino acid residues 1-313 of SEQ ID NO:5) can be prepared as a fusion to another soluble pHHLA2 dimerizing protein. Preferred dimerizing proteins in this regard include immunoglobulin constant region domains. Immunoglobulin-pHHLA2 polypeptide fusions can be expressed in genetically engineered cells to produce a variety of pHHLA2 analogs. Auxiliary domains can be fused to pHHLA2 polypeptides to target them to specific cells, tissues, or macromolecules (e.g., collagen). A pHHLA2 polypeptide can be fused to two or more moieties, such as an affinity tag for purification and a targeting domain. Polypeptide fusions can also comprise one or more cleavage sites, particularly between domains. See, Tuan et al., Connective Tissue Research 34:1-9, 1996. Additionally, the soluble multimeric cytokine receptor may further include an affinity tag. An affinity tag can be, for example, a tag selected from the group of polyhistidine, protein A, glutathione S transferase, Glu-Glu, substance P, Flag™ peptide, streptavidin binding peptide, and an immunoglobulin F_{c} polypeptide.

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470-7476, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for pHHLA2 amino acid residues.

Essential amino acids in the polypeptides of the present invention can be identified according to procedures known in the art, such as site-directed mutagenesis or alanine-scanning mutagenesis (Cunningham and Wells, Science 244: 1081-5, 1989; Bass et al., Proc. Natl. Acad. Sci. USA 88:4498-502, 1991). In the latter technique, single alanine mutations are introduced at every residue in the molecule, and the resultant mutant molecules are tested for biological activity (e.g. ligand binding and signal transduction) as disclosed below to identify amino acid residues that are critical to the activity of the molecule. See also, Hilton et al., J. Biol. Chem. 271:4699-4708, 1996. Sites of ligand-receptor, protein-protein or other biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-312, 1992; Smith et al., J. Mol. Biol. 224:899-904, 1992; Wlodaver et al., FEBS Lett. 309:59-64, 1992. The identities of essential amino acids can also be inferred from analysis of homologies with related receptors.

Determination of amino acid residues that are within regions or domains that are critical to maintaining structural integrity can be determined. Within these regions one can determine specific residues that will be more or less tolerant of change and maintain the overall tertiary structure of the molecule. Methods for analyzing sequence structure include, but are not limited to, alignment of multiple sequences with high amino acid or nucleotide identity and computer analysis using available software (e.g., the Insight II® viewer and homology modeling tools; MSI, San Diego, Calif.), secondary structure propensities, binary patterns, complementary packing and buried polar interactions (Barton, Current Opin. Struct. Biol. 5:372-376, 1995 and Cordes et al., Current Opin. Struct. Biol. 6:3-10, 1996). In general, when designing modifications to molecules or identifying specific fragments determination of structure will be accompanied by evaluating activity of modified molecules.

Amino acid sequence changes are made in pHHLA2 polypeptides so as to minimize disruption of higher order structure essential to biological activity. For example, when the pHHLA2 polypeptide comprises one or more helices, changes in amino acid residues will be made so as not to disrupt the helix geometry and other components of the molecule where changes in conformation abate some critical function, for example, binding of the molecule to its binding partners. The effects of amino acid sequence changes can be predicted by, for example, computer modeling as disclosed above or determined by analysis of crystal structure (see, e.g., Lapthorn et al., Nat. Struct. Biol. 2:266-268, 1995). Other techniques that are well known in the art compare folding of a variant protein to a standard molecule (e.g., the native protein). For example, comparison of the cysteine pattern in a variant and standard molecules can be made. Mass spectrometry and chemical modification using reduction and alkylation provide methods for determining cysteine residues which are associated with disulfide bonds or are free of such associations (Bean et al., Anal. Biochem. 201:216-226, 1992; Gray, Protein Sci. 2:1732-1748, 1993; and Patterson et al., Anal. Chem. 66:3727-3732, 1994). It is generally believed that if a modified molecule does not have the same disulfide bonding pattern as the standard molecule folding would be affected. Another well known and accepted method for measuring folding is circular dichrosism (CD). Measuring and comparing the CD spectra generated by a modified molecule and standard molecule is routine (Johnson, Proteins 7:205-214, 1990). Crystallography is another well known method for analyzing folding and structure. Nuclear magnetic resonance (NMR), digestive peptide mapping and epitope mapping are also known methods for analyzing folding and structural similarities between proteins and polypeptides (Schaanan et al., Science 257:961-964, 1992).

A Hopp/Woods hydrophilicity profile of the pHHLA2 polypeptide sequence as shown in SEQ ID NO:2 and SEQ ID NO:5 can be generated (Hopp et al., Proc. Natl. Acad. Sci. 78:3824-3828, 1981; Hopp, J. Immun. Meth. 88:1-18, 1986 and Triquier et al., Protein Engineering 11:153-169, 1998). The profile is based on a sliding six-residue window. Buried G, S, and T residues and exposed H, Y, and W residues were ignored.

Those skilled in the art will recognize that hydrophilicity or hydrophobicity will be taken into account when designing modifications in the amino acid sequence of a pHHLA2 polypeptide, so as not to disrupt the overall structural and biological profile. Of particular interest for replacement are hydrophobic residues selected from the group consisting of Val, Leu and lie or the group consisting of Met, Gly, Ser, Ala, Tyr and Trp. For example, residues tolerant of substitution could include such residues as shown in SEQ ID NO:2 and SEQ ID NO:5. However, Cysteine residues would be relatively intolerant of substitution.

The identities of essential amino acids can also be inferred from analysis of sequence similarity between B7 family members with pHHLA2. Using methods such as “FASTA” analysis described previously, regions of high similarity are identified within a family of proteins and used to analyze amino acid sequence for conserved regions. An alternative approach to identifying a variant pHHLA2 polynucleotide on the basis of structure is to determine whether a nucleic acid molecule encoding a potential variant pHHLA2 polynucleotide can hybridize to a nucleic acid molecule having the nucleotide sequence of SEQ ID NO:1 or SEQ ID NO:4, as discussed herein.

Other methods of identifying essential amino acids in the polypeptides of the present invention are procedures known in the art, such as site-directed mutagenesis or alanine-scanning mutagenesis (Cunningham and Wells, Science 244:1081 (1989), Bass et al., Proc. Natl. Acad. Sci. USA 88:4498 (1991), Coombs and Corey, “Site-Directed Mutagenesis and Protein Engineering,” in Proteins: Analysis and Design, Angeletti (ed.), pages 259-311 (Academic Press, Inc. 1998)). In the latter technique, single alanine mutations are introduced at every residue in the molecule, and the resultant mutant molecules are tested for biological activity as disclosed below to identify amino acid residues that are critical to the activity of the molecule. See also, Hilton et al., J. Biol. Chem. 271:4699 (1996).

The present invention also includes a soluble pHHLA2 polypeptide which includes functional fragments of soluble pHHLA2 polypeptides and nucleic acid molecules encoding such functional fragments. Routine deletion analyses of nucleic acid molecules can be performed to obtain functional fragments of a nucleic acid molecule that encodes a pHHLA2 polypeptide. As an illustration, DNA molecules having the nucleotide sequence of SEQ ID NO:1 or SEQ ID NO:5 or fragments thereof, can be digested with Bal31 nuclease to obtain a series of nested deletions. These DNA fragments are then inserted into expression vectors in proper reading frame, and the expressed polypeptides are isolated and tested for pHHLA2 activity, or for the ability to bind anti-pHHLA2. One alternative to exonuclease digestion is to use oligonucleotide-directed mutagenesis to introduce deletions or stop codons to specify production of a desired pHHLA2 fragment. Alternatively, particular fragments of a pHHLA2 polynucleotide can be synthesized using the polymerase chain reaction.

Standard methods for identifying functional domains are well-known to those of skill in the art. For example, studies on the truncation at either or both termini of interferons have been summarized by Horisberger and Di Marco, Pharmac. Ther. 66:507 (1995). Moreover, standard techniques for functional analysis of proteins are described by, for example, Treuter et al., Molec. Gen. Genet. 240:113 (1993); Content et al., “Expression and preliminary deletion analysis of the 42 kDa 2-5A synthetase induced by human interferon,” in Biological Interferon Systems, Proceedings of ISIR-TNO Meeting on Interferon Systems, Cantell (ed.), pages 65-72 (Nijhoff 1987); Herschman, “The EGF Receptor,” in Control of Animal Cell Proliferation 1, Boynton et al., (eds.) pages 169-199 (Academic Press 1985); Coumailleau et al., J. Biol. Chem. 270:29270 (1995); Fukunaga et al., J. Biol. Chem. 270:25291 (1995); Yamaguchi et al., Biochem. Pharmacol. 50:1295 (1995); and Meisel et al., Plant Molec. Biol. 30:1 (1996).

Multiple amino acid substitutions can be made and tested using known methods of mutagenesis and screening, such as those disclosed by Reidhaar-Olson and Sauer (Science 241:53-57, 1988) or Bowie and Sauer (Proc. Natl. Acad. Sci. USA 86:2152-2156, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-10837, 1991; Ladner et al., U.S. Pat. No. 5,223,409; Huse, WIPO Publication WO 92/062045) and region-directed mutagenesis (Derbyshire et al., Gene 46:145, 1986; Ner et al., DNA 7:127, 1988).

Variants of the disclosed pHHLA2 polynucleotide and polypeptide sequences can be generated through DNA shuffling as disclosed by Stemmer, Nature 370:389-91, 1994, Stemmer, Proc. Natl. Acad. Sci. USA 91:10747-51, 1994 and WIPO Publication WO 97/20078. Briefly, variant DNAs are generated by in vitro homologous recombination by random fragmentation of a parent DNA followed by reassembly using PCR, resulting in randomly introduced point mutations. This technique can be modified by using a family of parent DNAs, such as allelic variants or DNAs from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed herein can be combined with high-throughput, automated screening methods to detect activity of cloned, mutagenized pHHLA2 co-receptor polypeptides in host cells. Preferred assays in this regard include cell proliferation assays and biosensor-based ligand-binding assays, which are described below. Mutagenized DNA molecules that encode active receptors or portions thereof (e.g., ligand-binding fragments, signaling domains, and the like) can be recovered from the host cells and rapidly sequenced using modern equipment. These methods allow the rapid determination of the importance of individual amino acid residues in a polypeptide of interest, and can be applied to polypeptides of unknown structure.

Using the methods discussed herein, one of ordinary skill in the art can identify and/or prepare a variety of polypeptide fragments or variants of SEQ ID NO:2 and SEQ ID NO:5 that retain the co-stimulating activity. For example, one can make a pHHLA2 “soluble receptor” by preparing a variety of polypeptides that are substantially homologous to the extracellular domain (residues 23 (Ile) to 346 (Gly) of SEQ ID NO:2; and residues 1 (Met) to 313 (Gly) of SEQ ID NO:5), or allelic variants or species orthologs thereof and retain inhibition of co-stimulating activity of the wild-type pHHLA2 protein. Such polypeptides may include additional amino acids from, for example, part or all of the transmembrane and intracellular domains. Such polypeptides may also include additional polypeptide segments as generally disclosed herein such as labels, affinity tags, and the like.

For any pHHLA2 polypeptide, which include variants, soluble receptors, and fusion polypeptides or proteins, one of ordinary skill in the art can readily generate a fully degenerate polynucleotide sequence encoding that variant using the information set forth in Tables 1 and 2 above.

The pHHLA2 polypeptides of the present invention, including full-length polypeptides, soluble polypeptides, functional fragments, and fusion polypeptides, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, John Wiley and Sons, Inc., NY, 1987.

The present invention also provides an expression vector comprising an isolated and purified DNA molecule including the following operably linked elements: a transcription promoter, a first DNA segment encoding a polypeptide having at least 95 percent sequence identity with amino acid residues 23-346 of SEQ ID NO:2 or amino acid residues 1-313 of SEQ ID NO:5, and a transcription terminator; wherein the encoded soluble polypeptide inhibits or antagonizes the costimulation of T cells. The DNA molecule may further comprise a secretory signal sequence operably linked to the DNA segment. The DNA segment may encode for a soluble co-receptor and may further encode an affinity tag. The present invention also provides a cultured cell containing the above-described expression vector.

In general, a DNA sequence, for example, encoding a pHHLA2 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct, for example, a pHHLA2 polypeptide into the secretory pathway of a host cell, a secretory signal sequence (also known as a leader sequence, prepro sequence or pre sequence) is provided in the expression vector. The secretory signal sequence may be that of pHHLA2, or may be derived from another secreted protein (e.g., t-PA) or synthesized de novo. The secretory signal sequence is operably linked to the pHHLA2 DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain secretory signal sequences may be positioned elsewhere in the DNA sequence of interest (see, e.g., Welch et al., U.S. Pat. No. 5,037,743; Holland et al., U.S. Pat. No. 5,143,830).

Alternatively, the secretory signal sequence contained in the polypeptides of the present invention is used to direct other polypeptides into the secretory pathway. The present invention provides for such fusion polypeptides. A signal fusion polypeptide can be made wherein a secretory signal sequence derived from amino acid 1 (Met) to amino acid 22 (Gly) of SEQ ID NO:2, is operably linked to another polypeptide using methods known in the art and disclosed herein. The secretory signal sequence contained in the fusion polypeptides of the present invention is preferably fused amino-terminally to an additional peptide to direct the additional peptide into the secretory pathway. Such constructs have numerous applications known in the art. For example, these novel secretory signal sequence fusion constructs can direct the secretion of an active component of a normally non-secreted protein. Such fusions may be used in vivo or in vitro to direct peptides through the secretory pathway.

The present invention also provides a cultured cell comprising a first expression vector comprising a DNA molecule containing the following operably linked elements: a transcription promoter, a DNA segment encoding a soluble polypeptide having at least 95 percent sequence identity with amino acid residues 23-346 of SEQ ID NO:2 or amino acid residues 1-313 of SEQ ID NO:5, and a transcription terminator; wherein the encoded soluble polypeptide inhibits the costimulation of T cells. The DNA segment may encode a soluble polypeptide that may be a homodimer or heterodimer, and/or may further comprise an affinity tag as described herein. The DNA segment may encode a full-length pHHLA2 polypeptide.

Cultured mammalian cells are suitable hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-845, 1982), DEAE-dextran mediated transfection (Ausubel et al., ibid.), and liposome-mediated transfection (Hawley-Nelson et al., Focus 15:73, 1993; Ciccarone et al., Focus 15:80, 1993, and viral vectors (Miller and Rosman, BioTechniques 7:980-90, 1989; Wang and Finer, Nature Med. 2:714-716, 1996). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, Rockville, Md. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g., hygromycin resistance, multi-drug resistance, puromycin acetyltransferase) can also be used. Alternative markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). See, King, L. A. and Possee, R. D., The Baculovirus Expression System: A Laboratory Guide, London, Chapman & Hall; O'Reilly, D. R. et al., Baculovirus Expression Vectors: A Laboratory Manual, New York, Oxford University Press., 1994; and, Richardson, C. D., Ed., Baculovirus Expression Protocols. Methods in Molecular Biology, Totowa, N.J., Humana Press, 1995. A second method of making recombinant pHHLA2 baculovirus utilizes a transposon-based system described by Luckow (Luckow, V. A, et al., J Virol 67:4566-79, 1993). This system, which utilizes transfer vectors, is sold in the Bac-to-Bac™ kit (Life Technologies, Rockville, Md.). This system utilizes a transfer vector, pFastBac1™ (Life Technologies) containing a Tn7 transposon to move the DNA encoding the pHHLA2 polypeptide into a baculovirus genome maintained in E. coli as a large plasmid called a “bacmid.” See, Hill-Perkins, M. S, and Possee, R. D., J Gen Virol 71:971-6, 1990; Bonning, B. C. et al., J Gen Virol 75:1551-6, 1994; and, Chazenbalk, G. D., and Rapoport, B., J. Biol Chem 270:1543-9, 1995. In addition, transfer vectors can include an in-frame fusion with DNA encoding an epitope tag at the C- or N-terminus of the expressed pHHLA2 polypeptide, for example, a Glu-Glu epitope tag (Grussenmeyer, T. et al., Proc. Natl. Acad. Sci. 82:7952-4, 1985). Using a technique known in the art, a transfer vector containing pHHLA2 is transformed into E. coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g., Sf9 cells. Recombinant virus that expresses pHHLA2 is subsequently produced. Recombinant viral stocks are made by methods commonly used in the art.

The recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994. Another suitable cell line is the High FiveO™ cell line (Invitrogen) derived from Trichoplusia ni (U.S. Pat. No. 5,300,435). Commercially available serum-free media are used to grow and maintain the cells. Suitable media are Sf900 II™ (Life Technologies) or ESF 921™ (Expression Systems) for the Sf9 cells; and Ex-cellO405™ (JRH Biosciences, Lenexa, Kans.) or Express FiveO™ (Life Technologies) for the T. ni cells. Procedures used are generally described in available laboratory manuals (King, L. A. and Possee, R. D., ibid.; O'Reilly, D. R. et al., ibid.; Richardson, C. D., ibid.). Subsequent purification of the pHHLA2 polypeptide from the supernatant can be achieved using methods described herein.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in Saccharomyces cerevisiae is the POTI vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-3465, 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

The use of Pichia methanolica as host for the production of recombinant proteins is disclosed in WIPO Publications WO 97/17450, WO 97/17451, WO 98/02536, and WO 98/02565. DNA molecules for use in transforming P. methanolica will commonly be prepared as double-stranded, circular plasmids, which are preferably linearized prior to transformation. For polypeptide production in P. methanolica, it is preferred that the promoter and terminator in the plasmid be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. A preferred selectable marker for use in Pichia methanolica is a P. methanolica ADE2 gene, which encodes phosphoribosyl-5-aminoimidazole carboxylase (AIRC; EC 4.1.1.21), which allows ade2 host cells to grow in the absence of adenine. For large-scale, industrial processes where it is desirable to minimize the use of methanol, it is preferred to use host cells in which both methanol utilization genes (AUG1 and AUG2) are deleted. For production of secreted proteins, host cells deficient in vacuolar protease genes (PEP4 and PRB1) are preferred. Electroporation is used to facilitate the introduction of a plasmid containing DNA encoding a polypeptide of interest into P. methanolica cells. It is preferred to transform P. methanolica cells by electroporation using an exponentially decaying, pulsed electric field having a field strength of from 2.5 to 4.5 kV/cm, preferably about 3.75 kV/cm, and a time constant (t) of from 1 to 40 milliseconds, most preferably about 20 milliseconds.

Prokaryotic host cells, including strains of the bacteria Escherichia coli, Bacillus and other genera are also useful host cells within the present invention. Techniques for transforming these hosts and expressing foreign DNA sequences cloned therein are well known in the art (see, e.g., Sambrook et al., ibid.). When expressing a pHHLA2 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell. P. methanolica cells are cultured in a medium comprising adequate sources of carbon, nitrogen and trace nutrients at a temperature of about 25° C. to 35° C. Liquid cultures are provided with sufficient aeration by conventional means, such as shaking of small flasks or sparging of fermentors. A preferred culture medium for P. methanolica is YEPD (2% D-glucose, 2% Bacto™ Peptone (Difco Laboratories, Detroit, Mich.), 1% Bacto™ yeast extract (Difco Laboratories), 0.004% adenine and 0.006% L-leucine).

Within one aspect of the present invention, a pHHLA2 polypeptide is produced by a cultured cell, and the cell is used to screen for its counterpart co-receptor on T cells. To summarize this approach, a cDNA or gene encoding the receptor is combined with other genetic elements required for its expression (e.g., a transcription promoter), and the resulting expression vector is inserted into a host cell. Cells that express the DNA and produce functional receptor are selected and used within a variety of screening systems.

pHHLA2 proteins of the present invention may be expressed in mammalian cells. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61; CHO DG44 (Chasin et al., Som. Cell. Molec. Genet. 12:555, 1986)), rat pituitary cells (GHI; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650) and murine embryonic cells (NIH-3T3; ATCC CRL 1658).

A soluble pHHLA2 polypeptide (e.g., monomer or homodimer) can be expressed as a fusion with an immunoglobulin heavy chain constant region, typically an F_{c} fragment, which contains two constant region domains and lacks the variable region. Methods for preparing such fusions are disclosed in U.S. Pat. Nos. 5,155,027 and 5,567,584. Such fusions are typically secreted as multimeric molecules wherein the F_{c} portions are disulfide bonded to each other and two non-Ig polypeptides are arrayed in closed proximity to each other. Fusions of this type can be used for example, for dimerization, increasing stability and in vivo half-life, to affinity purify ligand, as in vitro assay tool or antagonist. For use in assays, the chimeras are bound to a support via the F_{c} region and used in an ELISA format.

The present invention also provides an antibody (e.g., neutralizing monoclonal antibodies, agonist monoclonal antibodies, polyclonal antibodies) that specifically binds to a pHHLA2 polypeptide or at least at portion thereof as described herein.

pHHLA2 polypeptide can also be used to prepare antibodies that bind to epitopes, peptides or polypeptides thereof (e.g., portion of the extracellular domain of SEQ ID NO:2 and/or SEQ ID NO:5). The extracellular domain of the pHHLA2 polypeptide or a fragment thereof serves as an antigen (immunogen) to inoculate an animal and elicit an immune response. One of skill in the art would recognize that antigenic, epitope-bearing polypeptides may contain a sequence of at least 6, preferably at least 9, and more preferably at least 15 to about 30 contiguous amino acid residues of the extracellular domain of the pHHLA2 polypeptide, such as amino acid residues 23-346 of SEQ ID NO:2 and/or amino acid residues 1-313 of SEQ ID NO:5. Polypeptides comprising a larger portion of a pHHLA2 polypeptide, e.g., from 30 to 100 residues up to the entire length of the amino acid sequence are included. Antigens or immunogenic epitopes can also include attached tags, adjuvants, carriers and vehicles, as described herein.

Antibodies from an immune response generated by inoculation of an animal with these antigens can be isolated and purified as described herein. Methods for preparing and isolating polyclonal and monoclonal antibodies are well known in the art. See, for example, Current Protocols in Immunology, Cooligan, et al. (eds.), National Institutes of Health, John Wiley and Sons, Inc., 1995; Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982.

As would be evident to one of ordinary skill in the art, polyclonal antibodies can be generated from inoculating a variety of warm-blooded animals such as horses, cows, goats, sheep, dogs, chickens, rabbits, mice, and rats with a pHHLA2 polypeptide or a fragment thereof. The immunogenicity of a multimeric cytokine receptor may be increased through the use of an adjuvant, such as alum (aluminum hydroxide) or Freund's complete or incomplete adjuvant. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

As used herein, the term “antibodies” includes polyclonal antibodies, affinity-purified polyclonal antibodies, monoclonal antibodies (e.g., neutralizing and agonsist), and antigen-binding fragments, such as F(ab′)₂ and Fab proteolytic fragments. Genetically engineered intact antibodies or fragments, such as “Fab fragment” (V_{L}-C_{L}-C_{H}I-V_{H}), “Fab′ fragment” (a Fab with the heavy chain hinge region) and “F(ab′)₂ fragment” (a dimer of Fab′ fragments joined by the heavy chain hinge region), chimeric antibodies, Fv fragments, single chain antibodies and the like, as well as synthetic antigen-binding peptides and polypeptides, are also included. Recombinant methods have been used to generate even smaller antigen-binding fragments, referred to as “single chain Fv” (variable fragment) or “scFv”, consisting of V_{L} and V_{H} joined by a synthetic peptide linker. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced. Moreover, human antibodies can be produced in transgenic, non-human animals that have been engineered to contain human immunoglobulin genes as disclosed in WIPO Publication WO 98/24893. It is preferred that the endogenous immunoglobulin genes in these animals be inactivated or eliminated, such as by homologous recombination.

Antibodies are considered to be specifically binding if: 1) they exhibit a threshold level of binding activity, and 2) they do not significantly cross-react with related polypeptide molecules. A threshold level of binding is determined if anti-multimeric cytokine receptor antibodies herein bind to a multimeric cytokine receptor, peptide or epitope with an affinity at least 10-fold greater than the binding affinity to control (non-multimeric cytokine receptor) protein. It is preferred that the antibodies exhibit a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art, for example, by Scatchard analysis (Scatchard, G., Ann. NY Acad. Sci. 51: 660-672 (1949)).

Whether anti-pHHLA2 polypeptide antibodies do not significantly cross-react with related polypeptide molecules is shown, for example, by the antibody detecting pHHLA2 polypeptide but not known related polypeptides using a standard Western blot analysis (Ausubel et al., ibid.). Examples of known related polypeptides are those disclosed in the prior art, such as known orthologs, and paralogs, and similar known members of a protein family. Screening can also be done using non-human pHHLA2 polypeptide, and pHHLA2 mutant polypeptides. Moreover, antibodies can be “screened against” known related polypeptides, to isolate a population that specifically binds to the pHHLA2 polypeptide. For example, antibodies raised to pHHLA2 polypeptide are adsorbed to related polypeptides adhered to insoluble matrix; antibodies specific to pHHLA2 polypeptide will flow through the matrix under the proper buffer conditions. Screening allows isolation of polyclonal and monoclonal antibodies non-crossreactive to known closely related polypeptides (Antibodies: A Laboratory Manual, Harlow and Lane (eds.), Cold Spring Harbor Laboratory Press, 1988; Current Protocols in Immunology, Cooligan, et al. (eds.), National Institutes of Health, John Wiley and Sons, Inc., 1995). Screening and isolation of specific antibodies is well known in the art. See, Fundamental Immunology, Paul (eds.), Raven Press, 1993; Getzoff et al., Adv. in Immunol. 43: 1-98, 1988; Monoclonal Antibodies: Principles and Practice, Goding, J. W. (eds.), Academic Press Ltd, 1996; Benjamin et al., Ann. Rev. Immunol. 2: 67-101, 1984. Specifically binding anti-pHHLA2 polypeptide antibodies can be detected by a number of methods in the art, and disclosed below.

A variety of assays known to those skilled in the art can be utilized to detect antibodies which bind to pHHLA2 co-receptor proteins or polypeptides. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radioimmunoassay, radioimmuno-precipitation, enzyme-linked immunosorbent assay (ELISA), dot blot or Western blot assay, inhibition or competition assay, and sandwich assay. In addition, antibodies can be screened for binding to wild-type versus mutant pHHLA2 co-receptor protein or polypeptide.

Within another aspect the present invention provides an antibody produced by the method as disclosed above, wherein the antibody binds to at least a portion of the extracellular domain of a pHHLA2 polypeptide comprising amino acid residues 23-346 of SEQ ID NO:2 or amino acid residues 1-313 of SEQ ID NO:5. In one embodiment, the antibody disclosed above specifically binds to a polypeptide shown in SEQ ID NO:2 or SEQ ID NO:5. In another embodiment, the antibody can be a neutralizing monoclonal antibody, a neutralizing antibody fragment, such as one or more scFv antibody fragments targeting the extracellular domain of pHHLA2 (e.g., bispecific or trispecific antibody), or a polyclonal antibody.

Antibodies to pHHLA2 polypeptide may be used for tagging cells that express pHHLA2 polypeptide; for isolating pHHLA2 polypeptide by affinity purification; for diagnostic assays for determining circulating levels of pHHLA2 polypeptide; for detecting or quantitating soluble pHHLA2 polypeptide as a marker of underlying pathology or disease; in analytical methods employing FACS; for screening expression libraries; for generating anti-idiotypic antibodies; and as neutralizing antibodies or as antagonists to block pHHLA2 polypeptide activity in vitro and in vivo. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Antibodies herein may also be directly or indirectly conjugated to drugs, toxins, radionuclides and the like, and these conjugates used for in vivo diagnostic or therapeutic applications. Moreover, antibodies to multimeric cytokine receptor or fragments thereof may be used in vitro to detect denatured multimeric cytokine receptor or fragments thereof in assays, for example, Western Blots or other assays known in the art.

Suitable detectable molecules may be directly or indirectly attached to the pHHLA2 polypeptide or antibody, and include radionuclides, enzymes, substrates, cofactors, inhibitors, fluorescent markers, chemiluminescent markers, magnetic particles and the like. Suitable cytotoxic molecules may be directly or indirectly attached to the polypeptide or antibody, and include bacterial or plant toxins (for instance, diphtheria, toxin, saporin, Pseudomonas exotoxin, ricin, abrin and the like), as well as therapeutic radionuclides, such as iodine-131, rhenium-188 or yttrium-90 (either directly attached to the polypeptide or antibody, or indirectly attached through means of a chelating moiety, for instance). Multimeric cytokine receptors or antibodies may also be conjugated to cytotoxic drugs, such as adriamycin. For indirect attachment of a detectable or cytotoxic molecule, the detectable or cytotoxic molecule can be conjugated with a member of a complementary/anticomplementary pair, where the other member is bound to the polypeptide or antibody portion. For these purposes, biotin/streptavidin is an exemplary complementary/anticomplementary pair.

Polypeptide-toxin fusion proteins or antibody-toxin fusion proteins can be used for targeted cell or tissue inhibition or ablation (for instance, to treat cancer cells or tissues). Alternatively, if the polypeptide has multiple functional domains (i.e., an activation domain or a receptor binding domain, plus a targeting domain), a fusion protein including only the targeting domain may be suitable for directing a detectable molecule, a cytotoxic molecule or a complementary molecule to a cell or tissue type of interest. In instances where the domain only fusion protein includes a complementary molecule, the anti-complementary molecule can be conjugated to a detectable or cytotoxic molecule. Such domain-complementary molecule fusion proteins thus represent a generic targeting vehicle for cell/tissue-specific delivery of generic anti-complementary-detectable/cytotoxic molecule conjugates.

The present invention also provides peptidomimetic compounds that are designed based upon the amino acid sequences of the functional peptide fragments. Peptidomimetic compounds are synthetic compounds having a three-dimensional conformation (i.e., a “peptide motif”) that is substantially the same as the three-dimensional conformation of a selected peptide. The peptide motif provides the peptidomimetic compound with the ability to co-stimulate T cells in a manner qualitatively identical to that of the pHHLA2 functional peptide fragment from which the peptidomimetic was derived. Peptidomimetic compounds can have additional characteristics that enhance their therapeutic utility, such as increased cell permeability and prolonged biological half-life.

The peptidomimetics typically have a backbone that is partially or completely non-peptide, but with side groups that are identical to the side groups of the amino acid residues that occur in the peptide on which the peptidomimetic is based. Several types of chemical bonds, e.g., ester, thioester, thioamide, retroamide, reduced carbonyl, dimethylene and ketomethylene bonds, are known in the art to be generally useful substitutes for peptide bonds in the construction of protease-resistant peptidomimetics.

The methods of the present invention involve contacting a T cell with a pHHLA2 polypeptide molecule, or a functional fragment thereof, in order to co-stimulate or antagonize pHHLA2s function to co-stimulate the T cell. The contacting can occur before, during, or after activation of the T cell. Contacting of the T cell with the pHHLA2 co-receptor polypeptide will preferably be at substantially the same time as activation. Activation can be, for example, by exposing the T cell to an antibody that binds to the TCR or one of the polypeptides of the CD3 complex that is physically associated with the TCR. Alternatively, the T cell can be exposed to either an alloantigen (e.g., a MHC alloantigen) on, for example, an antigen presenting cell (APC) (e.g., a dendritic cell, a macrophage, a monocyte, or a B cell) or an antigenic peptide produced by processing of a protein antigen by any of the above APC and presented to the T cell by MHC molecules on the surface of the APC. The T cell can be a CD4+T cell or a CD8+T cell. The pHHLA2 co-receptor molecule can be added to the solution containing the cells, or it can be expressed on the surface of an APC, e.g., an APC presenting an alloantigen or an antigen peptide bound to an MHC molecule. Alternatively, if the activation is in vitro, the pHHLA2 co-receptor molecule can be bound to the floor of a the relevant culture vessel, e.g. a well of a plastic microtiter plate.

The methods can be performed in vitro, in vivo, or ex vivo. In vitro application of pHHLA2 co-receptor can be useful, for example, in basic scientific studies of immune mechanisms or for production of activated T cells for use in either studies on T cell function or, for example, passive immunotherapy. Furthermore, pHHLA2 co-receptor could be added to in vitro assays (e.g., in T cell proliferation assays) designed to test for immunity to an antigen of interest in a patient from which the T cells were obtained. Addition of pHHLA2 co-receptor to such assays would be expected to result in a more potent, and therefore more readily detectable, in vitro response.

The pHHLA2 co-receptor proteins and variants thereof are generally useful as immune response-stimulating therapeutics. For example, the polypeptides of the invention can be used for treatment of disease conditions characterized by immunosuppression: e.g., cancer, AIDS or AIDS-related complex, other virally or environmentally-induced conditions, and certain congenital immune deficiencies. The compounds may also be employed to increase immune function that has been impaired by the use of radiotherapy of immunosuppressive drugs such as certain chemotherapeutic agents, and therefore are particularly useful when given in conjunction with such drugs or radiotherapy.

These methods of the invention can be applied to a wide range of species or patients, e.g., humans, non-human primates, horses, cattle, pigs, sheep, goats, dogs, cats, rabbits, guinea pigs, hamsters, rats, and mice.

In the United States approximately 500,000 people suffer from Inflammatory Bowel Disease (IBD) which can affect either colon and rectum (Ulcerative colitis) or both, small and large intestine (Crohn's Disease). The pathogenesis of these diseases is unclear, but they involve chronic inflammation of the affected tissues. Potential therapeutics include pHHLA2 soluble polypeptides, which includes soluble fusion proteins), or anti-pHHLA2 antibodies or antibody fragments of the present invention, that could serve as a valuable therapeutic to reduce inflammation and pathological effects in IBD and related diseases.

Crohn's disease is a chronic disorder that causes inflammation of the digestive and gastrointestinal (GI) tract. Although it can involve any area of the GI tract from the mouth to the anus, it most commonly affects the small intestine and/or colon. Symptoms of Crohn's disease include diarrhea (loose, watery, or frequent bowel movements), crampy abdominal pain, fever, and, at time, rectal bleeding. These are the hallmark symptoms of Crohn's disease, but they may vary from person to person and may change over time. Loss of appetite and subsequent weight loss also may occur. Fatigue is a common condition. Some patients may develop tears (fissures) in the lining of the anus. Inflammation may also cause a fistula to develop. A fistula is a tunnel that leads from one loop of the intestine to another, or that connects the intestine to the bladder, vagina, or skin. Symptoms may range from mild to severe. Patients will go through periods in which the disease flares up, is active, and causes symptoms. These episodes are followed by times of remission—periods in which symptoms disappear or the severity of the disease decreases. Drugs used to treat Crohn's disease include aminosalicylates (5-ASA) (e.g., Asacol®, Colazal®, Dipentum®, or Pentasa®), corticosteroids (e.g., prednisone and methylprednisone), immune modifiers (e.g., azathioprine (Imuran®), 6-MP (Purinethol®), and methotrexateImmune modifiers), antibiotics (e.g., metronidazole, ampicillin, ciprofloxacin), and biologic therapies (e.g., infliximab (e.g., Remicade®).

Celiac disease is an autoimmune disorder of the digestive system that occurs in genetically-predisposed individuals. It is characterised by damage or flattening to all or part of the villi lining the small intestine, which interferes with the absoption of nutrients. This damage is caused by eating anything with gluten (gliadin), a protein found in wheat, rye, and barley. Gastrointestinal or digestive problems occur in some coeliacs. Some celiac patients suffer from diarrhea, weight loss, and nutritional deficiencies. Celiacs, however, can suffer from a wide range and severity of symptoms, which include everything from canker sores to diarrhea to constipation to nausea. Many of the symptoms may mimic other diseases such as irritable bowel syndrome, reflux, or even Crohn's disease and coeliac may be misdiagnosed as any of these. Other symptoms that may occur are bulky, pale, offensive-smelling stools which may float in the toilet bowl, excess flatulence, infrequent, minor rectal bleeding, or persistent pain in the abdomen. Some symptoms appear to be caused because the villi are unable to absorb nutrients. Some examples are osteoporosis, damage to teeth enamel, anemia, fatigue, rapid or unexplained weight loss, overweight, failure to thrive or stunted growth in children, etc. Yet other symptoms appear to be emotional, such as depression and irritability. Dermatitis herpetiformis is an itchy blistering skin disease that occurs in some coeliacs and is considered to be an external manifestation of coeliac disease. The only treatment is a life-long gluten-free diet.

Irritable bowel syndrome (IBS) or spastic colon is a functional bowel disorder characterized by abdominal pain and changes in bowel habits. There are various causes of the set of IBS symptoms, including food allergies and sensitivities. Argument continues on the definition of cause as regards IBS and food allergies, but studies demonstrate that IBS symptoms are sometimes caused by immune response to foods and exclusion of those foods to which the immune system is responding results in reduction or elimination of IBS symptoms, a cause and effect link.

Ulcerative colitis (UC) is an inflammatory disease of the large intestine, commonly called the colon, characterized by inflammation and ulceration of the mucosa or innermost lining of the colon. This inflammation causes the colon to empty frequently, resulting in diarrhea. Symptoms include loosening of the stool and associated abdominal cramping, fever and weight loss. Although the exact cause of UC is unknown, recent research suggests that the body's natural defenses are operating against proteins in the body which the body thinks are foreign (an “autoimmune reaction”). Perhaps because they resemble bacterial proteins in the gut, these proteins may either instigate or stimulate the inflammatory process that begins to destroy the lining of the colon. As the lining of the colon is destroyed, ulcers form releasing mucus, pus and blood. The disease usually begins in the rectal area and may eventually extend through the entire large bowel. Repeated episodes of inflammation lead to thickening of the wall of the intestine and rectum with scar tissue. Death of colon tissue or sepsis may occur with severe disease. The symptoms of ulcerative colitis vary in severity and their onset may be gradual or sudden. Attacks may be provoked by many factors, including respiratory infections or stress. The most common symptoms of UC are abdominal pain and diarrhea. UC patients may also experience anemia, fatigue, weight loss, loss of appetite, rectal bleeding, loss of body fluids and nutrients, skin lesions, joint pain and growth failure (especially in children).

Although there is currently no cure for UC available, treatments are focused on suppressing the abnormal inflammatory process in the colon lining. Treatments including corticosteroids (e.g., prednisone, methyprednisone and hydrocortisone), aminosalicylates (drugs that contain 5-aminosalicyclic acid (5-ASA) such as, for instance, sulfasalazine, olsalazine, mesalamine and balsalazide) and immunomodulators (e.g., azathioprine and 6-mercapto-purine are available to treat the ulcerative colitis. However, the long-term use of immunosuppressives such as corticosteroids and azathioprine can result in serious side effects including thinning of bones, cataracts, infection, and liver and bone marrow effects. In the patients in whom current therapies are not successful, surgery is an option. The surgery involves the removal of the entire colon and the rectum.

There are several animal models that can partially mimic chronic ulcerative colitis. The most widely used model is the 2,4,6-trinitrobenesulfonic acid/ethanol (TNBS) induced colitis model, which induces chronic inflammation and ulceration in the colon. When TNBS is introduced into the colon of susceptible mice via intra-rectal instillation, it induces T-cell mediated immune response in the colonic mucosa, in this case leading to a massive mucosal inflammation characterized by the dense infiltration of T-cells and macrophages throughout the entire wall of the large bowel. Moreover, this histopathologic picture is accompanies by the clinical picture of progressive weight loss (wasting), bloody diarrhea, rectal prolapse, and large bowel wall thickening (Neurath et al. Intern. Rev. Immunol. 19:51-62, 2000).

Another colitis model uses dextran sulfate sodium (DSS), which induces an acute colitis manifested by bloody diarrhea, weight loss, shortening of the colon and mucosal ulceration with neutrophil infiltration. DSS-induced colitis is characterized histologically by infiltration of inflammatory cells into the lamina propria, with lymphoid hyperplasia, focal crypt damage, and epithelial ulceration. These changes are thought to develop due to a toxic effect of DSS on the epithelium and by phagocytosis of lamina propria cells and production of TNF-alpha and IFN-gamma. Despite its common use, several issues regarding the mechanisms of DSS about the relevance to the human disease remain unresolved. DSS is regarded as a T cell-independent model because it is observed in T cell-deficient animals such as SCID mice.

Inflammation in the gut resulting from defective immune regulation, known as inflammatory bowel disease (IBD) is characterized into two broad disease definitions, Crohn's disease (CD) and Ulcerative colitis (UC). Additional inflammatory diseases of the gut resulting from defective immune regulation are celiac disease and Irritable Bowel Syndrome (IBS). Generally, CD is thought to be due to dysfunction in the regulation of Th1 responses, and UC is believed to be due to dysfunction in the regulation of Th2 responses. Multiple cytokines, chemokines, and matrix metaloproteinases have beens shown to be upregulated in inflamed lesions from IBD patients. These include IL-1, IL-12, IL-18, IL-15, TNF-α, IFN-γ, MIP1α, MIP1β, and MIP2. Currently REMICADE® (Centocor, Malvern, Pa.) is the only drug that has successfully been used to target the disease itself when treating CD patients, with other treatments generally improving the quality of life of patients. IL-28A, IL-28B, and IL-29 inhibition of the autoimmune response associated with IBD is demonstrated in IBD models, such as the mouse DSS, TNBS, CD4+ CD45Rbhi, mdrla−/− and graft v. host disease (GVHD) intestinal inflammation models. (Stadnicki A and Colman R W, Arch Immunol Ther Exp 51:149-155, 2003; Pizarro T T et al., Trends in Mol Med 9:218-222, 2003). One experimental model for human IBD is the oral administration of dextran sodium sulfate (DSS) to rodents. DSS induces both acute and chronic ulcerative colitis with features somewhat resembling histological findings in humans. Colitis induced by DSS involves gut bacteria, macrophages and neutrophils, with a minor role for T and B cells (Mahler et al., Am. J. Physiol. 274:G544-G551, 1998; Egger et al., Digestion 62:240-248, 2000). TNBS-induced colitis is considered a Th1 mediated disease and therefore resembles CD more than UC in humans. Tri-nitro benzene sulfonic acid (TNBS) is infused into mice intra-rectally in varying doses (strain dependent) to induce antigen specific (TNBS) T cell response that involves secretion of Th1-like cytokines IL-12, IL-18 and IFNγ. Colitis involves recruitment of antigen-specific T cells, macrophages and neutrophils to the site of inflammation (Neurath et al., Int. Rev. Immunol., 19:51-62, 2000; Dohi T et al., J. Exp. Med. 189:1169-1179, 1999). Another relatively new model for colitis is the CD4+ CD45RB^{hi} transfer model into SCID mice. CD4⁺ T cells can be divided broadly into 2 categories based on expression of CD45Rb. CD4+CD45RB^{hi} cells are considered naïve T cells whereas CD4+CD45Rb^{lo} cells are considered regulatory T cells. Transfer of whole CD4⁺ T cells into syngenic SCID mice does not induce symptoms of colitis. However, if only the CD4⁺CD45RB^{hi} T cells are injected into SCID mice, mice develop colitis over a period of 3-6 weeks. Co-transfer of the CD4+CD45Rb^{lo} regulatory T cells along with the naïve T cells inhibits colitis suggesting that the regulatory T cells play an important role in regulating the immune response (Leach et al., Am. J. Pathol., 148:1503-1515, 1996; Powrie et al., J. Exp. Med., 179:589-600, 1999). This model will demonstrate that pHHLA2 antagonists (pHHLA2 antibody or soluble pHHLA2 polypeptide) inhibit colitis by inhibiting the activation of T cells and the activated T cells to express and secrete inflammatory cytokines. A clinically relevant model of colitis associated with bone marrow transplantation is GVHD-induced colitis. Graft-versus-host disease (GVHD) develops in immunoincompetent, histocompatible recipients of effector cells, which proliferate and attack host cells. Patients receiving allogeneic bone marrow transplantation or severe aplastic anemia are at risk for GVHD. In both mice and humans, diarrhea is a common and serious symptom of the syndrome. In human, both colonic and small intestinal diseases have been observed. Mouse models for GVHD-induced colitis show similar histological disease as seen in humans. These mouse models can therefore be used to assess the efficacy of colitis inhibiting drugs for GVHD (Eigenbrodt et al., Am. J. Pathol., 137:1065-1076, 1990; Thiele et al., J. Clin. Invest., 84:1947-1956, 1989).

Accordingly, the present invention contemplates the use of a pHHLA2 antagonist (e.g., neutralizing antibody or fragment thereof and soluble/fusion pHHLA2 polypeptides, e.g., amino acid residues 23-346 of SEQ ID NO:2 or portion thereof, or amino acid residues 1-313 of SEQ ID NO:5 or portion thereof,) to treat, prevent, inhibit the progression of, delay the onset of, and/or reduce at least one of the symptoms or conditions associated a disease selected from the group of Crohn's disease, ulcerative colitis, celiac disease, Graft-versus-host disease, and irritable bowel syndrome. Another embodiment of the invention is to use in combination with the current treatment for Crohn's disease, ulcerative colitis, celiac disease and irritable bowel syndrome a pHHLA2 antagonist as described herein.

For purposes of therapy, molecules having pHHLA2 antagonistic (e.g., soluble pHHLA2 polypeptide, antibody or antibody fragment to pHHLA2) activity and a pharmaceutically acceptable vehicle are administered to a patient in a therapeutically effective amount. A combination of a protein, polypeptide, or peptide having pHHLA2 antagonistic activity and a pharmaceutically acceptable vehicle is said to be administered in a “therapeutically effective amount” or “effective amount” if the amount administered is physiologically significant. An agent is physiologically significant if its presence results in a detectable change in the physiology of a recipient patient. For example, an agent used to treat inflammation is physiologically significant if its presence alleviates at least a portion of the inflammatory response.

In one in vivo approach, the pHHLA2 co-receptor polypeptide (or a functional fragment thereof) itself is administered in a “therapeutically effective amount” to the patient. An amount is considered to be a “therapeutically effective amount” if its presence results in a detectable change in the physiology of a recipient subject. For example, an agent used to treat inflammation is physiologically significant if its presence alleviates at least a portion of the inflammatory response.

Generally, the compounds of the invention will be suspended in a pharmaceutically-acceptable carrier (e.g., physiological saline) and administered orally or by intravenous infusion, or injected subcutaneously, intramuscularly, intraperitoneally, intrarcctally, intravaginally, intranasally, intragastrically, intratracheally, or intrapulmonarily. They are preferably delivered directly to an appropriate lymphoid tissue (e.g. spleen, lymph node, or mucosal-associated lymphoid tissue (MALT)). The dosage required depends on the choice of the route of administration, the nature of the formulation, the nature of the patient's illness, the subject's size, weight, surface area, age, and sex, other drugs being administered, and the judgment of the attending physician. Suitable dosages are in the range of 0.01-100.0 .mu.g/kg. Wide variations in the needed dosage are to be expected in view of the variety of polypeptides and fragments available and the differing efficiencies of various routes of administration. For example, oral administration would be expected to require higher dosages than administration by i.v. injection. Variations in these dosage levels can be adjusted using standard empirical routines for optimization as is well understood in the art. Administrations can be single or multiple (e.g., 2- or 3-, 4-, 6-, 8-, 10-, 20-, 50-, 100-, 150-, or more fold). Encapsulation of the polypeptide in a suitable delivery vehicle (e.g., polymeric microparticles or implantable devices) may increase the efficiency of delivery, particularly for oral delivery.

Alternatively, a polynucleotide containing a nucleic acid sequence encoding the pHHLA2 polypeptide or functional fragment can be delivered to an appropriate cell of the animal. Expression of the coding sequence will preferably be directed to lymphoid tissue of the subject by, for example, delivery of the polynucleotide to the lymphoid tissue. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 .mu.m in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 .mu.m and preferably larger than 20 .mu.m.

An additional method to achieve uptake of the nucleic acid is using liposomes, prepared by standard methods. The vectors can be incorporated alone into these delivery vehicles or co-incorporated with tissue-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells (Cristiano et al. (1995), J. Mol. Med. 73, 479). Alternatively, lymphoid tissue specific targeting can be achieved by the use of lymphoid tissue-specific transcriptional regulatory elements (TRE) such as a B lymphocyte, T lymphocyte, or dendritic cell specific TRE. Lymphoid tissue specific TRE are known (Thompson et al. (1992), Mol. Cell. Biol. 12, 1043-1053; Todd et al. (1993), J. Exp. Med. 177, 1663-1674; Penix et al. (1993), J. Exp. Med. 178, 1483-1496). Delivery of “naked DNA” (i.e., without a delivery vehicle) to an intramuscular, intradermal, or subcutaneous site, is another means to achieve in vivo expression.

Peripheral blood mononuclear cells (PBMC) can be withdrawn from the patient or a suitable donor and exposed ex vivo to an activating stimulus and a pHHLA2 co-receptor polypeptide or polypeptide fragment (whether in soluble form or attached to a sold support by standard methodologies). The PBMC containing highly activated T cells are then introduced into the same or a different patient.

An alternative ex vivo strategy can involve transfecting or transducing cells obtained from the subject with a polynucleotide encoding a pHHLA2 co-receptor polypeptide or functional fragment-encoding nucleic acid sequences described above. The transfected or transduced cells are then returned to the patient. While such cells would preferably be hemopoietic cells (e.g., bone marrow cells, macrophages, monocytes, dendritic cells, or B cells) they could also be any of a wide range of types including, without limitation, fibroblasts, epithelial cells, endothelial cells, keratinocytes, or muscle cells in which they act as a source of the pHHLA2 co-receptor polypeptide or functional fragment for as long as they survive in the subject. The use of hemopoietic cells, that include the above APC, would be particular advantageous in that such cells would be expected to home to, among others, lymphoid tissue (e.g., lymph nodes or spleen) and thus the pHHLA2 co-receptor polypeptide or functional fragment would be produced in high concentration at the site where they exert their effect, i.e., enhancement of an immune response. In addition, if APC are used, the APC expressing the exogenous pHHLA2 co-receptor molecule can be the same APC that presents an alloantigen or antigenic peptide to the relevant T cell. The pHHLA2 co-receptor can be secreted by the APC or expressed on its surface. Prior to returning the recombinant APC to the patient, they can optionally be exposed to sources of antigens or antigenic peptides of interest, e.g., those of tumors, infectious microorganisms, or autoantigens. The same genetic constructs and trafficking sequences described for the in vivo approach can be used for this ex vivo strategy. Furthermore, tumor cells, preferably obtained from a patient, can be transfected or transformed by a vector encoding a pHHLA2 co-receptor polypeptide or functional fragment thereof. The tumor cells, preferably treated with an agent (e.g., ionizing irradiation) that ablates their proliferative capacity, are then returned to the patient where, due to their expression of the exogenous pHHLA2 co-receptor (on their cell surface or by secretion), they can stimulate enhanced tumoricidal T cell immune responses. It is understood that the tumor cells which, after transfection or transformation, are injected into the patient, can also have been originally obtained from an individual other than the patient.

The ex vivo methods include the steps of harvesting cells from a patient, culturing the cells, transducing them with an expression vector, and maintaining the cells under conditions suitable for expression of the pHHLA2 co-receptor polypeptide or functional fragment. These methods are known in the art of molecular biology. The transduction step is accomplished by any standard means used for ex vivo gene therapy, including calcium phosphate, lipofection, electroporation, viral infection, and biolistic gene transfer. Alternatively, liposomes or polymeric microparticles can be used. Cells that have been successfully transduced are then selected, for example, for expression of the coding sequence or of a drug resistance gene. The cells may then be lethally irradiated (if desired) and injected or implanted into the patient.

The invention provides methods for testing compounds (small molecules or macromolecules) that inhibit or enhance an immune response. Such a method can involve, e.g., culturing a pHHLA2 co-receptor of the invention (or a functional fragment thereof with T cells in the presence of a T cell stimulus (see above). The pHHLA2 co-receptor molecule can be in solution or membrane bound (e.g., expressed on the surface of the T cells) and it can be natural or recombinant. Compounds that inhibit the T cell response will likely be compounds that inhibit an immune response while those that enhance the T cell response will likely be compounds that enhance an immune response.

The invention also relates to using pHHLA2 co-receptor or functional fragments thereof to screen for immunomodulatory compounds that can interact with pHHLA2 co-receptor. One of skill in the art would know how to use standard molecular modeling or other techniques to identify small molecules that would bind to T cell interactive sites of pHHLA2 co-receptor. One such example is provided in Broughton (1997) Curr. Opin. Chem. Biol. 1, 392-398.

A candidate compound whose presence requires at least 1.5-fold (e.g., 2-fold, 4-fold, 6-fold, 10-fold, 150-fold, 1000-fold, 10,000-fold, or 100,000-fold) more B7-H1 in order to achieve a defined arbitrary level of T cell activation than in the absence of the compound can be useful for inhibiting an immune response. On the other hand, a candidate compound whose presence requires at least 1.5 fold (e.g., 2-fold, 4-fold, 6-fold, 10-fold, 100-fold, 1000-fold, 10,000 fold, or 100,000-fold) less pHHLA2 co-receptor to achieve a defined arbitrary level of T cell activation than in the absence of the compound can be useful for enhancing an immune response. Compounds capable of interfering with or modulating pHHLA2 co-receptor function are good candidates for immunosuppressive immunoregulatory agents, e.g., to modulate an autoimmune response or suppress allogeneic or xenogeneic graft rejection.

The present invention is further illustrated by the following non-limiting examples.

### EXAMPLES

### Example 1

### Construction of Expression Vector Human pHHLA2Avi-HIS TagpZMP21

In the effort to create the tetramer molecules an expression plasmid containing a polynucleotide encoding the extra-cellular domain of human pHHLA2, the Avi Tag and His Tag was constructed. A DNA fragment of the extra-cellular domain of human pHHLA2 was isolated by PCR using the polynucleotide sequence of SEQ ID NO:7 with flanking regions at the 5′ and 3′ ends corresponding to the vector sequence and the Avi Tag and HIS Tag sequences flanking the human pHHLA2 insertion point SEQ ID NOs:8 and 9, respectively. The primers zc50487 and zc50736 are shown in SEQ ID NOs:10 and 11, respectively.

The PCR reaction mixture was run on a 2% agarose gel and a band corresponding to the size of the insert was gel-extracted using a QIAquick™ Gel Extraction Kit (Qiagen, Valencia, Calif.). Plasmid pZMP21 is a mammalian expression vector containing an expression cassette having the MPSV promoter, multiple restriction sites for insertion of coding sequences, a stop codon, an E. coli origin of replication; a mammalian selectable marker expression unit comprising an SV40 promoter, enhancer and origin of replication, a DHFR gene, and the SV40 terminator; and URA3 and CEN-ARS sequences required for selection and replication in S. cerevisiae. It was constructed from pZP9 (deposited at the American Type Culture Collection, 10801 University Boulevard, Manassas, Va. 20110-2209, under Accession No. 98668) with the yeast genetic elements taken from pRS316 (deposited at the American Type Culture Collection, 10801 University Boulevard, Manassas, Va. 20110-2209, under Accession No. 77145), an internal ribosome entry site (IRES) element from poliovirus, and the extracellular domain of CD8 truncated at the C-terminal end of the transmembrane domain. Plasmid pZMP21 was digested with EcoRI/BglII to cleave off the PTA leader and used for recombination with the PCR insert.

The recombination was performed using the BD In-Fusion™ Dry-Down PCR Cloning kit (BD Biosciences, Palo Alto, Calif.). The mixture of the PCR fragment and the digested vector in 10 μl was added to the lyophilized cloning reagents and incubated at 37° C. for 15 minutes and 50° C. for 15 minutes. The reaction was ready for transformation. Two microliters of recombination reaction was transformed into One Shot TOP10 Chemical Competent Cells (Invitrogen, Carlbad, Calif.); the transformation was incubated on ice for 10 minutes and heat shocked at 42° C. for 30 seconds. The reaction was incubated on ice for 2 minutes (helping transformed cells to recover). After two minutes of incubation, 300 μl of SOC (2% Bacto™ Tryptone (Difco, Detroit, Mich.), 0.5% yeast extract (Difco), 10 mM NaCl, 2.5 mM KCl, 10 mM MgCl₂, 10 mM MgSO₄, 20 mM glucose) was added and the transformation was incubated at 37° C. with shaker for one hour. The whole transformation was plated on one LB AMP plates (LB broth (Lennox), 1.8% Bacto™ Agar (Difco), 100 mg/L Ampicillin).

The colonies were screened by PCR using primers zc50487 (SEQ ID NO:10) and zc50736 (SEQ ID NO:11). The positive colonies were verified by sequencing. The correct construct was designated as hHHLA2AviHISpZMP21.

### Example 2

### Construction of Expression Vector Human pHHLA2mFc2pZMP21

A pZMP21 expression plasmid containing a function extracellular domain of human pHHLA2×1 (1 (Met) to 344 (Asn) of SEQ ID NO:2 or 1 (Met) to 344 (Asn) of SEQ ID NO:12) fused to mouse Fc2 (345 (Glu) to 437 (Pro) of SEQ ID NO:12) was constructed (SEQ ID NO:12). A pHHLA2 PCR fragment was generated using primers zc48957 (SEQ ID NO:13) and zc48958 (SEQ ID NO:14) using clonetrack CT:101518 as template as follows: 1 cycle, 94° C., 2 minutes; 30 cycles, 94° C., 1 minute, followed by 55° C., 1 minute, followed by 72° C., 2 minutes; 1 cycle, 72° C., 10 minutes. The PCR reaction mixture was run on a 1% agarose gel and a band corresponding to the sizes of the inserts were gel-extracted using a QIAquick™ Gel Extraction Kit (Qiagen, Cat. No. 28704). The purified PCR fragment was subsequently digested with EcoRI and BglII and again band purified as described above. The resulting fragment was ligated into pZMP21 inserted gene/mFc2 that had been cut with EcoRI and BglII to eliminate the inserted gene and allow for insertion of the pHHLA2×1 gene in frame with mFc2. Two microliters of the above ligation was electroporated into electromax DH10B (25 uF/30 ohms/2100 volts/2 mm gap cuvette). Clones from this ligation were screened for insert by digestion with BamHI and three clones with the appropriate 1.802 kB insert were submitted to sequencing. All three clones submitted to sequencing had various point mutations, but two clones were found that could be pieced together to make a clone of the correct sequence. Clone #4413 and #4414 were cut with Mfel and the appropriate bands were purified and religated. One microliter of the above ligation was electroporated into electromax DH10B (25 uF/30 ohms/2100 volts/2 mm gap cuvette). Clones were screened by EcoRI and BglII digestion and two clones with the appropriate 1.048 kB insert were submitted to DNA sequencing. One of these clones (#4445) was found to be sequence correct (SEQ ID NO:12). The polynucleotide sequence of SEQ ID NO:12 encodes the pHHLA2mFc2 fusion protein. The polynucleotide sequence of SEQ ID NO:12 encodes a first N-terminal portion (same as amino acid residues 1 to 344 of SEQ ID NO:2 while having two silent mutations (caC, not caT, encodes Histidine at position 72 of SEQ ID NO:12 and tcG, not tcA, encodes Serine at position 105 of SEQ ID NO:12) and a second C-terminal portion (mouse Fc2-345 (Glu) to 437 (Pro) of SEQ ID NO:12).

### Example 3

### Purification and Analysis of pHHLA2mFc2 from CHO Cells

A. Purification of pHHLA2mFc2

The expression vector human pHHLA2mFc2pZMP21 (Example 2) was transfected into Chinese Hamster Ovary (CHO) cells. The CHO transfection was performed using methods known in the art. Approximately 10 L of conditioned media was harvested and sterile filtered using Nalgene 0.2 μm filters.

Protein was purified from the filtered media by a combination of Poros A50 Protein A affinity chromatography (PerSeptive Biosystems, 1-5559-01, Framingham, Mass.) and Superdex 200 size exclusion chromatography (Amersham Pharmacia Biotech, Piscataway, N.J.). A 118 ml Poros A50 Protein A column (50 mm×60 mm) was pre-eluted with 3 column volumes (CV) of 25 mM Sodium Citrate-Sodium Phosphate, 250 mM Ammonium Sulfate pH 3 buffer and equilibrated with 20 CV PBS pH 7.2. The CHO culture supernatant was 0.2 μm filtered and adjusted to pH 7.2. Direct loading to the Protein A column at 31 cm/hr overnight at 4° C. captured the pHHLA2mFc2 in the adjusted supernatant. After loading was complete, the column was washed with 10 CV of equilibration buffer. Next the column was washed with 10 CV of 25 mM Sodium Citrate-Sodium Phosphate, 250 mM Ammonium Sulfate pH 7.2 buffer and then the bound protein was eluted at 62 cm/hr with a 5 CV gradient from pH 7.2 to pH 3 formed using the Citrate-Phosphate-Ammonium Sulfate buffers. Some aggregated material eluted early but the bulk of the pHHLA2mFc2 eluted from the column at approximately pH 4.8. Fractions of 10 ml each were collected into tubes containing 600 μl of 2.0 M Tris, pH 8.0, in order to neutralize the eluted proteins. The fractions were pooled based on A280 and non-reducing SDS-PAGE. pHHLA2mFc2-containing fractions were pooled and concentrated to 10 ml by ultrafiltration in an Amicon Ultra-15 30K NWML centrifugal device (Millipore), and injected onto a 318 ml (26 mm×300 mm) Superdex 200 column pre-equilibrated in 35 mM Sodium Phosphate, 120 mM NaCl pH 7.3 at 28 cm/hr. The fractions containing purified pHHLA2mFc2 were pooled based on A280 and SDS PAGE, filtered through a 0.2 μm filter and frozen as aliquots at −80° C. The concentration of the final purified protein was determined by BCA assay (Pierce, Rockford, Ill.).

B. Analysis of Purified pHHLA2mFc2

Recombinant pHHLA2mFc2 was analyzed by SDS-PAGE (4-12% BisTris, Invitrogen, Carlsbad, Calif.) with 0.1% Coomassie R250 staining for protein and by immunoblotting with Anti-murine-IgG-HRP. The purified protein was electrophoresed using an Invitrogen Novex's Xcell II mini-cell, and transferred to nitrocellulose (0.2 mm; Invitrogen, Carlsbad, Calif.) at ambient temperature at 600 mA for 45 minutes in a buffer containing 25 mM Tris base, 200 mM glycine, and 20% methanol. The filters were then blocked with 10% non-fat dry milk in 50 mM Tris, 150 mM NaCl, 5 mM EDTA, 0.05% Igepal (TBS) for 15 minutes at room temperature. The nitrocellulose was quickly rinsed, and the IgG-HRP antibody (1:10,000) was added in. The blots were incubated overnight at 4° C., with gentle shaking. Following the incubation, the blots were washed three times for 10 minutes each in TBS, and then quickly rinsed in H₂O. The blots were developed using commercially available chemiluminescent substrate reagents (Roche LumiLight), and the signal was captured using Lumi-Imager's Lumi Analyst 3.0 software (Boehringer Mannheim GmbH, Germany). The purified pHHLA2mFc2 appeared as a single band on both the Western blot and the either Coomassie stained gel at about 180 kDa under non-reducing conditions, and at about 90 kDa under reducing conditions, suggesting a glycosylated dimeric form under non-reducing conditions as expected. The protein had the correct NH₂ terminus, the correct amino acid composition, and the correct mass by SEC MALS. The overall process recovery was 65-70%.

### Example 4

### pHHLA2 Monoclonal Antibodies

Female BALB/c mice were immunized with either pHHLA2/pVAC2 (extracellular domain of pHHLA2×1-amino acid residues 1-344 of SEQ ID NO:2)(Invivogen, San Diego, Calif.) DNA or P815 cells (ATCC, Manassas, Va.) expressing pHHLA2 (extracellular domain of pHHLA2×1-amino acid residues 1-344 of SEQ ID NO:2). Mice with positive serum titers were given a prefusion boost of soluble pHHLA2mFc2 fusion protein (Example 2).

Splenocytes were harvested from three high-titer mice and fused to P3-X63-Ag8/ATCC (mouse) myeloma cells in 3 separate fusion procedures using PEG 1500 (Roche Applied Science, Indianapolis, Ind.). Fusion 321 and 323 used spleen and lymph nodes from genetically immunized mice, while Fusion 322 pooled spleen, lymph and mesenteric nodes from a cell immunized mouse. Following 12 days growth post-fusion, specific antibody-producing hybridoma pools were identified using direct ELISA, capture ELISA, and FMAT (Applied Biosystems) screening. Both ELISA formats used purified recombinant pHHLA2-Avi-His tagged protein as the specific antibody target, while FMAT screening tested binding of antibody to P815 cells expressing pHHLA2. Fifty masterwells with positive assay results from at least one screen were chosen to keep, and were further analyzed via FACS for the ability to bind P815/pHHLA2 cells. From these, five were chosen to clone twice by limiting dilution. Clones were screened using capture ELISA and FACS analysis, which correlated directly.

Five final clones were harvested and purified for use in assays:

E9346, E9347, E9348, E9349 and E9350

### Example 5

### T Cell Proliferation is Enhanced by pHHLA2 on Transfected Cells

The proliferation of purified CD4 and CD8 T cells from human peripheral blood mononuclear cells (PBMC) is enhanced by pHHLA2 transfected into FDC cells. Antibody to CD3 (BD Biosciences 555329) mimics T cell antigen recognition. Engagement of CD3 and the T cell receptor by antibody provides a signal to proliferate in vitro. This signal can be significantly enhanced by a second, or co-stimulatory, signal.

Artificial antigen presenting cells (APC) were constructed to test the ability of pHHLA2 to provide a co-stimulatory signal to T cells. FDC cells were transfected by Lipofectamine 2000 (Invitrogen) with full-length pHHLA2×1 (pzmp21) and mouse zcyto35 (SEQ ID NO:15) (pzmp21) as a negative control. FDC were γ-irradiated with 10,000 rads to inhibit their proliferation in vitro. 5×10E4 FDC were plated per 96 well, flat bottom tissue culture plates.

Human PBMC from healthy volunteers were collected by Ficoll-Paque (GE Healthcare) density gradient. CD4 and CD8 were co-purified from PBMC by magnetic bead columns (Miltenyi Biotec). T cells were labeled with CFSE (Invitrogen) to assess proliferation by flow cytometry. 2×10E5 CFSE-labeled T cells were plated per well. Anti-CD3 was added to each well in soluble form over a range from 50 ng/ml to 1 ug/ml. Cultures were maintained for 3 days in humidified incubators at 5% CO2. Proliferation of CD4s and CD8s was assessed on an LSRII (Becton Dickinson).

T cell cultures with FDC-pHHLA2 proliferated extensively compared to T cell cultures with FDC-mycto35 (>70% compared to <10% of all T cells fell in the proliferating gate respectively). CD4s and CD8s were similar in response. There was no observed donor-to-donor variability.

pHHLA2 enhancement of T Cell Proliferation is Inhibited by Specific Monoclonal Antibody

Monoclonal antibodies to pHHLA2 inhibited the co-stimulatory effect provided by pHHLA2 on transfected cells. CD4 and CD8 T cells were collected and labeled with CFSE. 1×10E5 T cells were plated per well. FDC were γ-irradiated and plated at 2.5×10E4 per well. Five anti-pHHLA2 monoclonals (Example 4-E9346, E9347, E9348, E9349 and E9350) at 1 ug/ml were assayed for the ability to block in vitro T cell co-stimulation mediated by 100 ng/ml anti-CD3. CTLA4-Fc (R&D) was used as a control to assess the contribution of endogenous CD80/CD86 expressed by FDC to co-simulation. After 3 days in culture, T cell proliferation was determined by flow cytometry. pHHLA2 antibodies blocked a significant amount of proliferation as compared to control mIgG1 antibody (30-80%). CTLA4-Fc blocked virtually all T cell proliferation.

### Example 6

### Tissue Distribution of Human pHHLA2 in Tissue Panels and Primary Human Cells Using Northern Blot and LUMINEX®

A. Human pHHLA2 Tissue Distribution Using Northern Blot

Human Multiple Tissue Northern Blots (Human 12-lane MTN Blot I, II and III, Cancer Profiling Array) (Clontech) were probed to determine the tissue distribution of human pHHLA2 expression.

An approximately 620 bp PCR derived probe for pHHLA2 was amplified using oligonucleotides ZC49085 (SEQ ID NO:16) and ZC49091 (SEQ ID NO:17) as primers. The PCR amplification was carried out as follows: Cycling conditions were 1 cycle at 95° C. 5′, 35 cycles at 94° C. 10″, 62° C. 20″, 72° C. 30″, and one final cycle at 72° C. 7′, and a hold at 4° C.

Reactions were run in an agarose gel and fragments were purified using Qiagen gel purification columns (Qiagen, Valencia, Calif.) according to the manufacturer's instructions. The fragment was quantitated by a spectrophotometer reading. Fifty or twenty-five nanograms of fragment was labeled using Prime-It II reagents (Stratagene, La Jolla, Calif.) according to the manufacturer's instructions, and separated from unincorporated nucleotides using an S-200 microspin column (Amersham, Piscataway, N.J.) according to the manufacturer's protocol. Blots to be probed were prehybridized overnight at 55° C. in ExpressHyb (BD Biosciences, Clontech Palo Alto, Calif.) in the presence of 100 ug/ml salmon sperm DNA (Stratagene, La Jolla, Calif.) and 6 ug/ml cot-1 DNA (Invitrogen, Carlsbad, Calif.) which were boiled and snap-chilled prior to adding to the blots. Radiolabelled pHHLA2, salmon sperm DNA and cot-I DNA were mixed together and boiled 5′, followed by a snap chilling on ice. Final concentrations of the salmon sperm DNA and cot-1 DNA were as in the prehybridization step and the final concentration of radiolabelled pHHLA2 was 1×10⁶ cpm/ml. Blots were hybridized overnight in a roller oven at 55° C., then washed copiously at RT in 2×SSC, 0.1% SDS, with several buffer changes, then at 65° C. The final wash was at 65° C. in 0.1×SSC, 0.1% SDS. Blots were then exposed to film with intensifying screens for 10 days.

The Multiple Tissue Northern Blots were then probed with a transferrin receptor probe, generated as follows: sense primer zc10565 (SEQ ID NO:18) and antisense primer zc10651 (SEQ ID NO:19) were used in a 50 ul PCR reaction with 5 μl 10× Advantage 2 buffer, 1 μl Advantage 2 cDNA polymerase mix (BD Biosciences, Clontech, Palo Alto, Calif.), 5 μl 10× Redi-Load (Invitrogen, Carlsbad Calif.), 4 μl 2.5 mM dNTPs (Applied Biosystems, Foster City, Calif.), 1 μl each zc10565 (SEQ ID NO:18) and zc10651 (SEQ ID NO:19), and 5 μl Placenta Marathon™ cDNA (BD Biosciences, Clontech, Palo Alto, Calif.). Cycling conditions were one cycle at 94° C., 2′, 35 cycles of 94° C. 20″ 57° C. 20″ 72° C. 45″one cycle at 72° C. 7′, followed by a 4° C. hold. The reaction was run in an agarose gel and the fragment was purified using Qiagen gel purification columns (Qiagen, Valencia, Calif.) according to the manufacturer's instructions. The fragment was quantitated by a spectrophotometer reading. The transferrin receptor fragment was labeled and used to probe the Multiple Tissue Northern Blots and the Fetal Tissue Northern blot as described above. Blots were exposed to film with intensifying screens for 7 days.

Results of probing multiple tissue northern blots indicate that pHHLA2 mRNA is highly expressed in testis, small intestine and colon. Moderate to low expression was also observed in kidney, pancreas, stomach and trachea. The transferrin receptor control probing experiment shows the blots were of good quality and a low to moderately expressed control gene could be observed with a 10-day exposure. Additionally, in the Cancer Profiling Array, pHHLA2 mRNA is predominantly restricted to the small intestine, colon and rectum, with some expression found in pancreas, stomach and kidney. The expression of pHHLA2 is greater in normal, non-cancerous tissue than in the tumor samples for these tissues. These results indicated that pHHLA2 is predominantly expressed in tissues of the gastrointestinal tract and that the mRNA is not obviously increased in cancers of these tissues.

B. pHHLA2 mRNA Distribution in Primary Cel/S Using LUMINEX®

Annotation of the cell types and growth conditions that affect expression of the receptor is a useful means of elucidating its function and predicting a source of ligand. To that end a wide variety of tissue and cell types were surveyed by LUMINEX®. A panel of aRNAs from human tissues was screened for pHHLA2 mRNA expression using LUMINEX®. The panel was made in-house and contained 48 antisense RNA (aRNA) samples from various normal and autoimmune human tissues and is shown in Table 5, below. The aRNAs came from in-house tissue sources or in-house RNA preps. RNA from hematopoetic cell subsets was derived from normal human donors, purified by fluorescent cell sorting (FACSAria, Becton-Dickinson Cytometry Systems, Palo Alto, Calif.). Naive and memory T cells were isolated using antibodies to: CD4, CD8 and CD45RB. B cells, NK cells and monocytes were isolated using antibodies to CD19, CD56 and CD14, respectively. Macrophage and DC were generated in vitro from CD14 positive monocytes. All antibodies were obtained from BD Biosystems (Palo Alto, Calif.). Epithelial and endothelial cells were obtained from Cambrex (Hopkinton, Mass.) and grown in-house using conditions provided by the manufacturer. In some cases, cells were stimulated with the following, detailed in Table 5: Anti-CD3 (1 μg/ml) and anti-CD28 (5 μg/ml)(BD Biosystems, Palo Alto, Calif.), anti-CD40 and anti-IgM (1 μg/ml) (BD Biosystems, Palo Alto, Calif.) and IL-4 (5 ng/ml)(R&D Systems, Minneapolis, Minn.), IL-2, IL-21, hI10 1 ng/ml (R&D Systems, Minneapolis, Minn.), hIFNγ 50 ng/ml (R&D Systems, Minneapolis, Minn.), LPS 2 ug/ml (Sigma Chemicals, St. Louis, Mo.) or hTNFα 2 ng/ml (R&D Systems, Minneapolis, Minn.). Epithelial and endothelial cells were treated for the indicated times with an inflammatory stimulus that included: all of the following at the indicated concentrations: LPS 2 ug/ml (Sigma Chemicals, St. Louis, Mo.), hTNFα 6 ng/ml (R&D Systems, Minneapolis, Minn.), hIFNγ 50 ng/ml (R&D Systems, Minneapolis, Minn., hIL1β 2 ng/ml (R&D Systems, Minneapolis, Minn.), pI:C 10 ug/ml (Sigma Chemicals, St. Louis, Mo.) and huCpG 10 ug/ml.

For RNA generation, purified cell populations were lysed in RLT buffer (Qiagen, Valencia, Calif.), passed through Qiashredder columns (Qiagen, Valencia, Calif.), and RNA extracted using RNeasy mini kits (Qiagen, Valencia, Calif.). Samples were treated with DNase I in columns (RNase-free DNase set, Qiagen, Valencia, Calif.). RNA was quantified and its quality determined using an Agilent 2100 Bioanalyzer. Biotinylated aRNA was generated using Message Amp™ aRNA Amplification Kit (Ambion, Austin, Tex.) according to the manufacturer's instructions.

An oligonucleotide specific for pHHLA2 was generated comprising the sequence of SEQ ID NO:20. This oligonucleotide was coupled to fluorescent LUMINEX® microspheres according to the manufacturer's directions. Briefly, microspheres were resuspended by vortexing and sonication for 20 seconds, transferred to microfuge tubes, spun at 14000 rpm for 2 minutes and resuspended in 50 μL of 0.1M MES (pH 4.5). One μL of oligo (1 mM stock) and 2.5 μL EDC added to microspheres and the mix was incubated 30 minutes in the dark. This step was repeated twice. Labelled microspheres were washed twice, resuspended in 50 μL of TE (pH 8.0) and counted on a hemacytometer.

Oligonucleotide-coupled microspheres were hybridized to biotinylated aRNA and then analyzed on LUMINEX® 100× Map technology analyzer (Bio-Plex system, BioRAD, Hercules, Calif.) according to the manufacturer's directions. Briefly, microspheres were resuspended by vortex and sonication for 20 seconds and resuspended to 2500 microspheres per 40 μL in 1.5×TMAC Hybridization Buffer plus 20 μL TE (pH 8.0). Biotinylated aRNA (5 μg) was added to microspheres, the mixture heated to 60° C. and incubated five hours with gentle shaking. The mixture was transferred to a 96-well plate, washed twice and 75 μL of streptavidin-R-phycoerythrin (4 μg/ml) was added. This reaction was mixed by shaking for 10 minutes. Fifty microliters of this mixture was analyzed on the LUMINEX® 100 Analyzer according to the system manual.

LUMINEX® gene expression analysis was performed by comparing the fluorescent values for pHHLA-2 and control genes in numerous cellular populations and diseased tissues. Normalization for gene expression was calculated using any of several housekeeping genes, Clathrin (primer ZC50398, SEQ ID NO:21) being the preferred choice. Comparison of pHHLA-2 mRNA expression amongst the stated cells and tissues indicates that pHHLA-2 is preferentially expressed in colonic tissue, including high levels of expression in Ulcerative Colitis. Moderate levels of expression were also noted in activated neutrophils. Of 200 genes examined, pHHLA-2 was significantly over-expressed in Ulcerative Colitis compared to 98% of the remaining genes. This would confirm the results using the Multiple Tissue Northern analysis and Cancer Cell Profiling. Further, the data extends the Northern analysis to suggest a high level of expression in the specific human autoimmune disease, Ulcerative Colitis.

TABLE 5

| | # | | |
|---|---|---|---|
| Cell type | samples | Stimulation Conditions | Time |
| Naive CD4 T cells | 2 | None | 0 |
| Naive CD4 T cells | 2 | Anti-CD3 + Anti-CD28 | 4 |
| Naive CD4 T cells | 2 | Anti-CD3 + Anti-CD28 | 18 |
| Memory CD4 T cells | 2 | None | 0 |
| Memory CD4 T cells | 2 | Anti-CD3 + Anti-CD28 | 4 |
| Memory CD4 T cells | 2 | Anti-CD3 + Anti-CD28 | 18 |
| Naive CD8 T cells | 2 | None | 0 |
| Naive CD8 T cells | 2 | Anti-CD3 + Anti-CD28 | 4 |
| Naive CD8 T cells | 2 | Anti-CD3 + Anti-CD28 | 18 |
| Memory CD8 T cells | 2 | None | 0 |
| Memory CD8 T cells | 2 | Anti-CD3 + Anti-CD28 | 4 |
| Memory CD8 T cells | 2 | Anti-CD3 + Anti-CD28 | 18 |
| B cells | 2 | None | 0 |
| B cells | 2 | Anti-IgM + Anti-CD40 + IL-4 | 18 |
| Monocytes | 1 | None | 0 |
| Monocytes | 1 | LPS + IFN-g | 4 |
| Monocytes | 1 | LPS + IFN-g | 18 |
| Neutrophils | 1 | None | 0 |
| Neutrophils | 1 | LPS + MALP-2 + TNFa | 4 |
| Neutrophils | 1 | LPS + MALP-2 + TNFa | 18 |
| Macrophages | 1 | None | 0 |
| Macrophages | 1 | LPS + IFNg + IL-10 | 18 |
| Dendritic Cells | 1 | LPS + TNFa + pI:C + | 18 |
| | | Anti-CD40 | |
| Bone Marrow cells | 1 | None | 0 |
| Inflammed Tonsil | 1 | None | 0 |
| Ulcerative Colitis | 1 | None | 0 |
| Crohn's Disease | 1 | None | 0 |
| HUVEC | 1 | None | 0 |
| HUVEC | 1 | Inflammatory mixture | 4 |
| HUVEC | 1 | Inflammatory mixture | 18 |
| HMVEC-Lung | 1 | None | |
| HMVEC-Lung | 1 | Inflammatory mixture | 4 |
| HMVEC-Lung | 1 | Inflammatory mixture | 18 |
| HPAEC | 1 | None | |
| HPAEC | 1 | Inflammatory mixture | 4 |
| HPAEC | 1 | Inflammatory mixture | 18 |
| HCAEC | 1 | None | |
| HCAEC | 1 | Inflammatory mixture | 4 |
| HCAEC | 1 | Inflammatory mixture | 18 |
| NHBr | 1 | None | |
| NHBr | 1 | Inflammatory mixture | 4 |
| NHBr | 1 | Inflammatory mixture | 18 |
| NHEK | 1 | None | |
| NHEK | 1 | Inflammatory mixture | 4 |
| NHEK | 1 | Inflammatory mixture | 18 |

### Example 7

### In Vitro Intestinal Epithelium Model for IBD

Intestinal epithelium express low levels of HLA class II antigens on their surface and that increased expression of these molecules is considered to be associated with the manifestation of inflammatory conditions such as Inflammatory Bowel Disease (IBD—Crohn's disease and Ulcerative Colitis), graft versus host disease (GVHD) and celiac disease (Hershberg et al., J Clin Invest., 100(1):204-15 (Jul. 1, 1997)). Expression of HLA class II molecules is a prerequisite for cells that function as antigen presenting cells, suggesting that intestinal epithelium interacts with CD4+ T cells in the intestinal tract and regulates antigen-driven immune responses in the local environment. Given the plethora of antigens to which intestinal epithelium are exposed, and the fine balance that must be maintained between intestinal tolerance to innocuous antigens and adequate immune responses to pathogenic organisms, the ability of epithelial cells to regulate antigen presentation to intestinal T cells is critical to this balance.

pHHLA-2, as a member of the B7-family, plays a role in the co-stimulation of antigen-specific T cell responses. Initial analysis of pHHLA-2 mRNA expression by Northern blot (Example 6) has suggested prominent expression of HHLA2 in intestinal tissues. If pHHLA-2 is shown to be expressed on the surface of intestinal epithelial cells then pHHLA-2 would likely be involved in regulation of intestinal T cell responses driven by gut epithelium. To determine whether pHHLA2 regulates intestinal T cell responses, soluble pHHLA-2 or an antibody to pHHLA2 (e.g., extracellular domain or portion thereof) are tested for inhibition of the activation of antigen-specific T cells by epithelial cell lines (e.g., T84 and HT-29) by blocking the interaction of pHHLA-2 with it's putative counter structure on the T cell. In brief, epithelial cell lines are plated at 50,000 cells per well in a flat bottom 96-well plate overnight. Cells are then be pulsed with antigen at varying concentrations for 6 h at 37° C. After washing, antigen-specific and appropriately HLA-restricted T cells are added and co-cultured with epithelial cells for 24 hours in the presence or absence of soluble pHHLA-2 or antibody to pHHLA2. The supernatants will be collected for analysis of inflammatory cytokines, including IFNγ, TNFα, IL-1β, IL-2, IL-6, IL-12, IL-13, IL-17, IL-18, IL-21 and IL-23. Many of these cytokines have been shown to be over-expressed in human IBD samples and are therefore implicated in the initiation and perpetuation of the pro-inflammatory immune response in the gut. For T cell proliferation assays, the epithelial cells will be irradiated prior to pulsing with antigen and co-cultured with T cells for 72 hr. The cultures will then be pulsed with 3h-thymidine for a further 16 h before harvesting.

Activated T cells are a primary source of pro-inflammatory cytokines and studies have demonstrated that transfer of activated T cells can induce IBD in mice. Therefore, down-regulation of T cell activation and cytokine production by blocking co-stimulatory signals provided by pHHLA2 would inhibit the inappropriate inflammatory response associated with intestinal inflammatory diseases such as IBD, celiac disease and Irritable Bowel Syndrome (IBS).

The complete disclosure of all patents, patent applications, and publications, and electronically available material (e.g., GenBank amino acid and nucleotide sequence submissions) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. An isolated soluble pHHLA2 polypeptide comprising a sequence of amino acid residues having at least 95% sequence identity with amino acid residues 23-346 of SEQ ID NO:2 or amino acid residues 1-313 of SEQ ID NO:5, wherein the polypeptide inhibits the costimulation of T cells.

2. An isolated polynucleotide encoding a solube polypeptide wherein the encoded polypeptide comprises a sequence of amino acid residues having at least 95% sequence identity with amino acid residues 23-346 of SEQ ID NO:2 or amino acid residues 1-313 of SEQ ID NO:5, wherein the encoded polypeptide inhibits the costimulation T cells.

3. An isolated polynucleotide comprising nucleotides selected from the group consisting of 67-1038 of SEQ ID NO:1, 1-1038 of SEQ ID NO:1, 67-1095 of SEQ ID NO:1, 1-1095 of SEQ ID NO:1, 67-1242 of SEQ ID NO:1, 1-1242 of SEQ ID NO:1, 1-939 of SEQ ID NO:4, 1-996 of SEQ ID NO:4, and 1-1143 of SEQ ID NO:4.

4. An isolated polynucleotide that hybridizes to a polynucleotide of claim 3 under stringent conditions of hybridization in buffer containing 5×SSC, 5×Denhardt's, 0.5% SDS, 1 mg salmon sperm/25 mls of hybridization solution incubated at 65° C. overnight, followed by high stringency washing with 0.2×SSC/0.1% SDS at 65° C., wherein the isolated polynucleotide encodes a soluble polypeptide that inhibits the costimulation T cells.

5. An expression vector comprising the following operably linked elements:
a transcription promoter;
a DNA segment encoding a polypeptide of claim 1; and
a transcription terminator.

6. A cultured cell into which has been introduced an expression vector of claim 5, wherein the cell expresses the polypeptide encoded by the DNA segment.

7. A method of producing a polypeptide comprising:
culturing a cell into which has been introduced an expression vector of claim 5, wherein the cell expresses the polypeptide encoded by the DNA segment; and
recovering the expressed polypeptide.

8. An antibody or antibody fragment that specifically binds to a polypeptide of claim 1.

9. The antibody of claim 8, wherein the antibody is selected from the group consisting of a polyclonal antibody, a murine monoclonal antibody, a humanized antibody derived from a murine monoclonal antibody, an antibody fragment, neutralizing antibody, and a human monoclonal antibody.

10. The antibody fragment of claim 8, wherein the antibody fragment is selected from the group consisting of F(ab′), F(ab), F(ab′)₂, Fab′, Fab, Fv, scFv, and minimal recognition unit.

11. An anti-idiotype antibody comprising an anti-idiotype antibody that specifically binds to the antibody of claim 8.

12. A fusion protein comprising a polypeptide comprising a sequence of amino acid residues having at least 95% sequence identity with amino acid residues 23-346 of SEQ ID NO:2 or amino acid residues 1-313 of SEQ ID NO:5; and a polyalkyl oxide moiety, wherein the fusion protein inhibits the co-stimulation of T cells.

13. The fusion protein of claim 12 wherein the polyalkyl oxide moiety is polyethylene glycol.

14. The fusion protein of claim 13 wherein the polyethylene glycol is N-terminally or C-terminally attached to the polypeptide.

15. The fusion protein of claim 13 wherein the polyethylene glycol is mPEG propionaldehyde.

16. The fusion protein of claim 13 wherein the polyethylene glycol is branched or linear.

17. The fusion protein of claim 13 wherein the polyethylene glycol has a molecular weight of about 5 kD, 12 kD, 20 kD, 30 kD, 40 kD or 50 kD.

18. A fusion protein comprising a polypeptide comprising a sequence of amino acid residues having at least 95% sequence identity with amino acid residues 23-346 of SEQ ID NO:2 or amino acid residues 1-313 of SEQ ID NO:5; and an immunoglobulin heavy chain constant region, wherein the fusion protein inhibits the co-stimulation of T cells.

19. The fusion protein of claim 18 wherein the immunoglobulin heavy chain constant region is an Fc fragment.

20. The fusion protein of claim 18 wherein the immunoglobulin heavy chain constant region is an isotype selected from the group consisting of an IgG, IgM, IgE, IgA and IgD.

21. The fusion protein of claim 20 wherein the IgG isotype is IgG1, IgG2, IgG3, or IgG4.

22. A formulation comprising:
an isolated soluble polypeptide comprising a sequence of amino acid residues having at least 95% sequence identity with amino acid residues 23-346 of SEQ ID NO:2 or amino acid residues 1-313 of SEQ ID NO:5; and
pharmaceutically acceptable vehicle.

23. A kit comprising the formulation of claim 22.

24. A formulation comprising:
an antibody or antibody fragment according to claim 8; and
pharmaceutically acceptable vehicle.

25. A method of inhibiting the co-stimulation a T cell, the method comprising contacting the T cell with a soluble polypeptide, the sequence of which comprises a sequence having at least 95% identify with amino acid residues 23-346 of SEQ ID NO:2 or amino acid residues 1-313 of SEQ ID NO:5, wherein the polypeptide inhibits the co-stimulation of the T cell.

26. The method of claim 25, wherein the contacting comprises culturing the polypeptide with the T cell in vitro.

27. The method of claim 25, wherein the T cell is in a patient.

28. The method of claim 27 wherein the contacting comprises administering the polypeptide to the patient.

29. The method of claim 27 wherein the contacting comprises administering a nucleic acid encoding the polypeptide to the patient.

30. The method of claim 27 wherein comprising (a) providing a recombinant cell which is the progeny of a cell obtained from the patient and has been transfected or transformed ex vivo with a nucleic acid molecule encoding the polypeptide so that the cell expresses the polypeptide; and (b) administering the cell to the patient.

31. The method of claim 30 wherein the recombinant cell is an antigen presenting cell (APC) and expresses the polypeptide on its surface.

32. The method of claim 31 wherein prior to the administering, the APC is pulsed with an antigen or an antigenic peptide.

33. The method of claim 27 wherein the patient is suffering from an inflammatory disease selected from the group consisting of Crohn's disease, ulcerative colitis, graft versus host disease, celiac disease, and irritable bowel syndrome.

34. A method of treating, preventing, inhibiting the progression of, delaying the onset of and/or reducing at least one of the symptoms or conditions associated with a disease selected from the group consisting of Crohn's disease, ulcerative colitis, celiac disease, Graft-versus-host disease, and irritable bowel syndrome comprising administering to the patient an effective amount of the formulation of claim 22.

35. A method of treating, preventing, inhibiting the progression of, delaying the onset of and/or reducing at least one of the symptoms or conditions associated with a disease selected from the group consisting of Crohn's disease, ulcerative colitis, celiac disease, Graft-versus-host disease, and irritable bowel syndrome comprising administering to the patient an effective amount of the formulation of claim 24.


SEQUENCE LISTING (21 sequences)

SEQ ID NO: 1 - DNA, 1245 bp, Homo sapiens
  misc_feature (1)...(1245): pHHLA2x1
  CDS (1)...(1245)
atgaaggcacagacagcactgtctttcttcctcattctcataacatctctgagtggatctcaaggcatattccctttggctttcttcatttatgttcctatgaatgaacaaatcgtcattggaagacttgatgaagatataattctcccttcttcatttgagaggggatccgaagtcgtaatacactggaagtatcaagatagctataaggttcatagttactacaaaggcagtgaccatttggaaagccaagatcccagatatgcaaacaggacatcccttttctataatgagattcaaaatgggaatgcgtcactatttttcagaagagtaagccttctggacgaaggaatttacacctgctatgtaggaacagcaattcaagtgattacaaacaaagtggtgctaaaggtgggagtttttctcacacccgtgatgaagtatgaaaagaggaacacaaacagcttcttaatatgcagcgtgttaagtgtttatcctcgtccaattatcacgtggaaaatggacaacacacctatctctgaaaacaacatggaagaaacagggtctttggattctttttctattaacagcccactgaatattacaggatcaaattcatcttatgaatgtacaattgaaaattcactgctgaagcaaacatggacagggcgctggacgatgaaagatggccttcataaaatgcaaagtgaacacgtttcactctcatgtcaacctgtaaatgattatttttcaccaaaccaagacttcaaagttacttggtccagaatgaaaagtgggactttctctgtcctggcttactatctgagctcctcacaaaatacaattatcaatgaatcccgattctcatggaacaaagagctgataaaccagagtgacttctctatgaatttgatggatcttaatctttcagacagtggggaatatttatgcaatatttcttcggatgaatatactttacttaccatccacacagtgcatgtagaaccgagccaagaaacagcttcccataacaaaggcttatggattttggtgccctctgcgattttggcagcttttctgctgatttggagcgtaaaatgttgcagagcccagctagaagccaggaggagcagacaccctgctgatggagcccaacaagaaagatgttgtgtccctcctggtgagcgctgtcccagtgcacccgataatggcgaagaaaatgtgcctctttcaggaaaagtatag

SEQ ID NO: 2 - protein, 414 aa, Homo sapiens
  VARIANT (1)...(414): pHHLA2x1
MKAQTALSFFLILITSLSGSQGIFPLAFFIYVPMNEQIVIGRLDEDIILPSSFERGSEVVIHWKYQDSYKVHSYYKGSDHLESQDPRYANRTSLFYNEIQNGNASLFFRRVSLLDEGIYTCYVGTAIQVITNKVVLKVGVFLTPVMKYEKRNTNSFLICSVLSVYPRPIITWKMDNTPISENNMEETGSLDSFSINSPLNITGSNSSYECTIENSLLKQTWTGRWTMKDGLHKMQSEHVSLSCQPVNDYFSPNQDFKVTWSRMKSGTFSVLAYYLSSSQNTIINESRFSWNKELINQSDFSMNLMDLNLSDSGEYLCNISSDEYTLLTIHTVHVEPSQETASHNKGLWILVPSAILAAFLLIWSVKCCRAQLEARRSRHPADGAQQERCCVPPGERCPSAPDNGEENVPLSGKV

SEQ ID NO: 3 - DNA, 1242 bp, Artificial Sequence
  Degenerate polynucleotide encoding the polypeptide of SEQ ID NO:2
  variation (1)...(1242): n = A, T, G or C
atgaargcncaracngcnytnwsnttyttyytnathytnathacnwsnytnwsnggnwsncarggnathttyccnytngcnttyttyathtaygtnccnatgaaygarcarathgtnathggnmgnytngaygargayathathytnccnwsnwsnttygarmgnggnwsngargtngtnathcaytggaartaycargaywsntayaargtncaywsntaytayaarggnwsngaycayytngarwsncargayccnmgntaygcnaaymgnacnwsnytnttytayaaygarathcaraayggnaaygcnwsnytnttyttymgnmgngtnwsnytnytngaygarggnathtayacntgytaygtnggnacngcnathcargtnathacnaayaargtngtnytnaargtnggngtnttyytnacnccngtnatgaartaygaraarmgnaayacnaaywsnttyytnathtgywsngtnytnwsngtntayccnmgnccnathathacntggaaratggayaayacnccnathwsngaraayaayatggargaracnggnwsnytngaywsnttywsnathaaywsnccnytnaayathacnggnwsnaaywsnwsntaygartgyacnathgaraaywsnytnytnaarcaracntggacnggnmgntggacnatgaargayggnytncayaaratgcarwsngarcaygtnwsnytnwsntgycarccngtnaaygaytayttywsnccnaaycargayttyaargtnacntggwsnmgnatgaarwsnggnacnttywsngtnytngcntaytayytnwsnwsnwsncaraayacnathathaaygarwsnmgnttywsntggaayaargarytnathaaycarwsngayttywsnatgaayytnatggayytnaayytnwsngaywsnggngartayytntgyaayathwsnwsngaygartayacnytnytnacnathcayacngtncaygtngarccnwsncargaracngcnwsncayaayaarggnytntggathytngtnccnwsngcnathytngcngcnttyytnytnathtggwsngtnaartgytgymgngcncarytngargcnmgnmgnwsnmgncayccngcngayggngcncarcargarmgntgytgygtnccnccnggngarmgntgyccnwsngcnccngayaayggngargaraaygtnccnytnwsnggnaargtn

SEQ ID NO: 4 - DNA, 1146 bp, Homo sapiens
  misc_feature (1)...(1146): pHHLA2x2
  CDS (1)...(1146)
atgaatgaacaaatcgtcattggaagacttgatgaagatataattctcccttcttcatttgagaggggatccgaagtcgtaatacactggaagtatcaagatagctataaggttcacagttactacaaaggcagtgaccatttggaaagccaagatcccagatatgcaaacaggacatcccttttctataatgagattcaaaatgggaatgcgtcgctatttttcagaagagtaagccttctggacgaaggaatttatacctgctatgtaggaacagcaattcaagtgattacaaacaaagtggtgctaaaggtgggagtttttctcacacccgtgatgaagtatgaaaagaggaacacaaacagcttcttaatatgcagcgtgttaagtgtttatcctcgtccaattatcacgtggaaaatggacaacacacctatctctgaaaacaacatggaagaaacagggtctttggattctttttctattaacagcccactgaatattacaggatcaaattcatcttatgaatgtacaattgaaaattcactgctgaagcaaacatggacagggcgctggacgatgaaagatggccttcataaaatgcaaagtgaacacgtttcactctcatgtcaacctgtaaatgattatttttcaccaaaccaagacttcaaagttacttggtccagaatgaaaagtgggactttctctgtcctggcttactatctgagctcctcacaaaatacaattatcaatgaatcccgattctcatggaacaaagagctgataaaccagagtgacttctctatgaatttgatggatcttaatctttcagacagtggggaatatttatgcaatatttcttcggatgaatatactttacttaccatccacacagtgcatgtagaaccgagccaagaaacagcttcccataacaaaggcttatggattttggtgccctctgcgattttggcagcttttctgctgatttggagcgtaaaatgttgcagagcccagctagaagccaggaggagcagacaccctgctgatggagcccaacaagaaagatgttgtgtccctcctggtgagcgctgtcccagtgcacccgataatggcgaagaaaatgtgcctctttcaggaaaagtatag

SEQ ID NO: 5 - protein, 381 aa, Homo sapiens [sequence omitted]

SEQ ID NO: 6 - DNA, 1143 bp, Artificial Sequence
  Degenerate polynucleotide encoding the polypeptide of SEQ ID NO:5
  variation (1)...(1143): n = A, T, G or C
atgaaygarcarathgtnathggnmgnytngaygargayathathytnccnwsnwsnttygarmgnggnwsngargtngtnathcaytggaartaycargaywsntayaargtncaywsntaytayaarggnwsngaycayytngarwsncargayccnmgntaygcnaaymgnacnwsnytnttytayaaygarathcaraayggnaaygcnwsnytnttyttymgnmgngtnwsnytnytngaygarggnathtayacntgytaygtnggnacngcnathcargtnathacnaayaargtngtnytnaargtnggngtnttyytnacnccngtnatgaartaygaraarmgnaayacnaaywsnttyytnathtgywsngtnytnwsngtntayccnmgnccnathathacntggaaratggayaayacnccnathwsngaraayaayatggargaracnggnwsnytngaywsnttywsnathaaywsnccnytnaayathacnggnwsnaaywsnwsntaygartgyacnathgaraaywsnytnytnaarcaracntggacnggnmgntggacnatgaargayggnytncayaaratgcarwsngarcaygtnwsnytnwsntgycarccngtnaaygaytayttywsnccnaaycargayttyaargtnacntggwsnmgnatgaarwsnggnacnttywsngtnytngcntaytayytnwsnwsnwsncaraayacnathathaaygarwsnmgnttywsntggaayaargarytnathaaycarwsngayttywsnatgaayytnatggayytnaayytnwsngaywsnggngartayytntgyaayathwsnwsngaygartayacnytnytnacnathcayacngtncaygtngarccnwsncargaracngcnwsncayaayaarggnytntggathytngtnccnwsngcnathytngcngcnttyytnytnathtggwsngtnaartgytgymgngcncarytngargcnmgnmgnwsnmgncayccngcngayggngcncarcargarmgntgytgygtnccnccnggngarmgntgyccnwsngcnccngayaayggngargaraaygtnccnytnwsnggnaargtn

SEQ ID NO: 7 - DNA, 1041 bp, Homo sapiens
atgaaggcacagacagcactgtctttcttcctcattctcataacatctctgagtggatctcaaggcatattccctttggctttcttcatttatgttcctatgaatgaacaaatcgtcattggaagacttgatgaagatataattctcccttcttcatttgagaggggatccgaagtcgtaatacactggaagtatcaagatagctataaggttcatagttactacaaaggcagtgaccatttggaaagccaagatcccagatatgcaaacaggacatcccttttctataatgagattcaaaatgggaatgcgtcactatttttcagaagagtaagccttctggacgaaggaatttacacctgctatgtaggaacagcaattcaagtgattacaaacaaagtggtgctaaaggtgggagtttttctcacacccgtgatgaagtatgaaaagaggaacacaaacagcttcttaatatgcagcgtgttaagtgtttatcctcgtccaattatcacgtggaaaatggacaacacacctatctctgaaaacaacatggaagaaacagggtctttggattctttttctattaacagcccactgaatattacaggatcaaattcatcttatgaatgtacaattgaaaattcactgctgaagcaaacatggacagggcgctggacgatgaaagatggccttcataaaatgcaaagtgaacacgtttcactctcatgtcaacctgtaaatgattatttttcaccaaaccaagacttcaaagttacttggtccagaatgaaaagtgggactttctctgtcctggcttactatctgagctcctcacaaaatacaattatcaatgaatcccgattctcatggaacaaagagctgataaaccagagtgacttctctatgaatttgatggatcttaatctttcagacagtggggaatatttatgcaatatttcttcggatgaatatactttacttaccatccacacagtgcatgtagaaccgagccaagaaacagcttcccataacaaaggctta

SEQ ID NO: 8 - DNA, 45 bp, Artificial Sequence
  Avi Tag
ggtctgaacgacatcttcgaagctcagaaaatcgaatggcacgaa

SEQ ID NO: 9 - DNA, 18 bp, Artificial Sequence
  His Tag
catcaccatcaccatcac

SEQ ID NO: 10 - DNA, 44 bp, Artificial Sequence
  primer ZC50487
cacaggtgtccagggaattcgcaagatgaaggcacagacagcac

SEQ ID NO: 11 - DNA, 123 bp, Artificial Sequence
  primer ZC50736
aggcgcgcctctagattagtgatggtgatggtgatgtccaccagatccttcgtgccattcgattttctgagcttcgaagatgtcgttcagacctccaccagatcctaagcctttgttatggga

SEQ ID NO: 12 - DNA, 1313 bp, Artificial Sequence
  pHHLA2x1mFc2 fusion protein
  CDS (1)...(1313)
atgaaggcacagacagcactgtctttcttcctcattctcataacatctctgagtggatctcaaggcatattccctttggctttcttcatttatgttcctatgaatgaacaaatcgtcattggaagacttgatgaagatataattctcccttcttcatttgagaggggatccgaagtcgtaatacactggaagtatcaagatagctataaggttcacagttactacaaaggcagtgaccatttggaaagccaagatcccagatatgcaaacaggacatcccttttctataatgagattcaaaatgggaatgcgtcgctatttttcagaagagtaagccttctggacgaaggaatttacacctgctatgtaggaacagcaattcaagtgattacaaacaaagtggtgctaaaggtgggagtttttctcacacccgtgatgaagtatgaaaagaggaacacaaacagcttcttaatatgcagcgtgttaagtgtttatcctcgtccaattatcacgtggaaaatggacaacacacctatctctgaaaacaacatggaagaaacagggtctttggattctttttctattaacagcccactgaatattacaggatcaaattcatcttatgaatgtacaattgaaaattcactgctgaagcaaacatggacagggcgctggacgatgaaagatggccttcataaaatgcaaagtgaacacgtttcactctcatgtcaacctgtaaatgattatttttcaccaaaccaagacttcaaagttacttggtccagaatgaaaagtgggactttctctgtcctggcttactatctgagctcctcacaaaatacaattatcaatgaatcccgattctcatggaacaaagagctgataaaccagagtgacttctctatgaatttgatggatcttaatctttcagacagtggggaatatttatgcaatatttcttcggatgaatatactttacttaccatccacacagtgcatgtagaaccgagccaagaaacagcttcccataacgagcccagatctcccacaatcaagccctgtcctccatgcaaatgcccagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccat

SEQ ID NO: 13 - DNA, 34 bp, Artificial Sequence
  primer ZC48957
gcatgaattcgcaagatgaaggcacagacagcac

SEQ ID NO: 14 - DNA, 34 bp, Artificial Sequence
  primer ZC48958
atgcagatctgggctcgttatgggaagctgtttc

SEQ ID NO: 15 - DNA, 1308 bp, mus musculus
  CDS (1)...(1308)
  misc_feature (1)...(1308): mouse zcyto35
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggatctggcagtagtagaggcggctctggaagcggaggaagcggaggagccggaagtaaactgtggagcagggctgtgctcttccctgccgcccaccggccaaagaggtcctcatcactgccattgaacccagtcctgcagacctccctggaggaggtggagctgctctacgagttcctgctggccgaacttgagatcagccctgacctgcagatctccatcaaggacgaggagctggcctccttgcggaaggcctcagacttccgcaccgtctgcaacaacgtcatccccaagagcatcccagacatccgccggctcagcgccagcctctccagccaccctggcatcctcaagaaagaagactttgaaaggacagtgctgaccctggcctacacagcctaccgcacagccctgtcccacggccatcagaaggacatctgggcgcagtccctcgttagcctcttccaggccctgaggcacgacttgatgcgctcctcacagccgggagtacctcccggatccggtggacatcaccatcaccatcactaa

SEQ ID NO: 16 - DNA, 20 bp, Artificial Sequence
  oligonucleotide primer ZC49085
aatcgtcattggaagacttg

SEQ ID NO: 17 - DNA, 20 bp, Artificial Sequence
  oligonucleotide primer ZC49091
tgacatgagagtgaaacgtg

SEQ ID NO: 18 - DNA, 23 bp, Artificial Sequence
  oligonucleotide primer ZC10565
tttgcagaaaaggttgcaaatgc

SEQ ID NO: 19 - DNA, 20 bp, Artificial Sequence
  oligonucleotide primer ZC10651
agcttttctgcagcagctct

SEQ ID NO: 20 - DNA, 25 bp, Artificial Sequence
  oligonucleotide
gtgatttgcacgtatacatccagat

SEQ ID NO: 21 - DNA, 35 bp, Artificial Sequence
  primer ZC50398
caggccaagtggtgtcctcatgttgtgctgtatcc